(19) United States
(12) Patent Application Publication
(10) Pub. No.: US 2024/0074927 A1
(43) Pub. Date: Mar. 7, 2024

(54) SYSTEMS AND METHODS FOR CONTROLLING CONTINUOUS LATERAL ROTATION THERAPY

(71) Applicant: Hill-Rom Services, Inc., Batesville, IN (US)

(72) Inventors: Olivia Gubitose Boyce, Batesville, IN (US); Jacqueline Burris, Batesville, IN (US); Brian Hoffman, Batesville, IN (US); Frank Sauser, Batesville, IN (US)

(73) Assignee: Hill-Rom Services, Inc., Batesville, IN (US)

(21) Appl. No.: 18/459,718

(22) Filed: Sep. 1, 2023

Related U.S. Application Data
(60) Provisional application No. 63/403,411, filed on Sep. 2, 2022.

(51) Int. Cl. A61G 7/057; A61G 7/018

(52) U.S. Cl. CPC A61G 7/05769; A61G 7/018; A61G 2203/42

(57) ABSTRACT

Disclosed herein are person support apparatuses for continuous lateral rotation therapy. The apparatus includes a person support surface configured to support a subject, the person support surface including an actuating device for rotating the person support surface, one or more sensors configured to detect parameters of the subject and the person support surface and generate sensor data indicative of the same, and a controller. The controller is operable to cause movement of the person support surface into the predetermined angle of rotation and determine, based on the sensor data, whether the subject is in the predetermined angle of rotation. If the subject is not in the predetermined angle of rotation, the controller generates and transmits a correction control signal to the actuating device to cause the person support surface to move the subject into the predetermined angle of rotation.

### CROSS-REFERENCE TO RELATED APPLICATION

The present application claims the priority benefit of U.S. Provisional Application Ser. No. 63/403,411, entitled “SYSTEMS AND METHODS FOR CONTROLLING CONTINUOUS LATERAL ROTATION THERAPY” and filed on Sep. 2, 2022, the entire contents of which are hereby incorporated by reference.

### TECHNICAL FIELD

The present disclosure relates to a system and method for controlling person support surfaces and, more particularly, to systems and methods for providing therapy via a person support surface.

### BACKGROUND

Currently, caregivers face significant challenges taking care of subjects on person support surfaces of a person support apparatus with therapy needs. Oftentimes, therapy includes lateral rotation of the person support surface for subjects needing pulmonary therapy or decubitus prevention or treatment. The person support surface is typically laterally rotated by an air support system having a plurality of air bladders including a right rotation bladder and a left rotation bladder. A pneumatic control system includes an air handling unit that is mounted to a base of the person support surface and a control device mounted, for example, to an end rail or side rail of the person support surface. The rotation bladders are inflated and deflated to rotate the person support surface, for example, to a left or right angle, to thereby turn the subject pursuant to a continuous lateral rotation therapy program. In practice, however, inflation or deflation of the rotation bladders might not result in the desired turning of the subject. Accordingly, a need exists for improved subject support surfaces that rotate the subject as prescribed by a particular continuous lateral rotation therapy program.

### SUMMARY

In one embodiment, a person support apparatus for continuous lateral rotation therapy includes a person support surface configured to support a subject, the person support surface including an actuating device for rotating the person support surface from a flat orientation to a rotated position; one or more sensors configured to detect one or more parameters of the subject and the person support surface and generate sensor data based on the one or more parameters thereof; and a controller. The controller is operable to access a database comprising at least one predetermined angle of rotation associated with one or more continuous lateral rotation therapy routines; retrieve a control signal for causing the actuating device to move the person support surface into the predetermined angle of rotation; transmit the control signal to the actuating device to cause the person support surface to move; determine, based on the sensor data, whether the subject is in the predetermined angle of rotation; if the subject is in the predetermined angle of rotation, update the database to indicate that the control signal moved the person support surface to the predetermined angle of rotation; if the subject is not in the predetermined angle of rotation, generate and transmit a correction control signal to the actuating device to cause the person support surface to move; determine, based on the sensor data, whether the correction control signal moved the subject into the predetermined angle of rotation; and if the subject is in the predetermined angle of rotation, update the database to indicate that the control signal and the correction control signal moved the person support surface into the predetermined angle of rotation.

In another embodiment, a person support apparatus includes a person support surface configured to support a subject, the person support surface including an actuating device for rotating the person support surface, from a flat orientation to either a rotation position; at least one first sensor configured to detect SpO2 of the subject and to generate SpO2 data; at least one second sensor configured to detect an actual angle of rotation of the subject and generate data indicative of the actual angle of rotation of the subject; and a controller. The controller is operable to control the actuating device, the controller comprising a processor and a non-transitory memory device, the memory device storing: a database comprising at least one predetermined angle of rotation associated with one or more continuous lateral rotation therapy routines and comprising at least one predetermined SpO2 range; and instructions that, when executed by the processor, cause the processor to: determine, based on the database, a control signal that will cause the actuating device to move the person support surface into the predetermined angle of rotation; transmit the control signal to the actuating device to thereby move the person support surface; determine, based on the data indicative of the actual angle, whether the subject is in the predetermined angle of rotation; determine, based on the SpO2 data, whether the subject is within the predetermined SpO2 range; and update the database to indicate whether, when moved into the predetermined angle of rotation, the subject was within the predetermined SpO2 range.

In yet another embodiment, a controller for a person support apparatus is provided, wherein the person support apparatus includes an actuating device for rotating the person support surface and one or more sensors configured to detect one or more parameters of a subject on the person support surface and generate sensor data based on the one or more parameters. The controller is operable to access a database comprising at least one predetermined angle of rotation associated with one or more continuous lateral rotation therapy routines; retrieve a control signal for causing the actuating device to move the person support surface into the predetermined angle of rotation; transmit the control signal to the actuating device to cause the person support surface to move; determine, based on the sensor data, whether the subject is in the predetermined angle of rotation; if the subject is in the predetermined angle of rotation, update the database to indicate that the control signal moved the person support surface to the predetermined angle of rotation; if the subject is not in the predetermined angle of rotation, generate and transmit a correction control signal to the actuating device to cause the person support surface to move; determine, based on the sensor data, whether the correction control signal moved the subject into the predetermined angle of rotation; and if the subject is in the predetermined angle of rotation, update the database to indicate that the control signal and the correction control signal moved the person support surface into the predetermined angle of rotation.

In yet another embodiment, a method of controlling a person support apparatus is provided, wherein the person support apparatus includes an actuating device for rotating the person support surface and one or more sensors configured to detect one or more parameters of a subject on the person support surface and generate sensor data based on the one or more parameters. The method includes accessing a database comprising at least one predetermined angle of rotation associated with one or more continuous lateral rotation therapy routines; retrieving a control signal for causing the actuating device to move the person support surface into the predetermined angle of rotation; transmitting the control signal to the actuating device to cause the person support surface to move; determining, based on the sensor data, whether the subject is in the predetermined angle of rotation; if the subject is in the predetermined angle of rotation, updating the database to indicate that the control signal moved the person support surface to the predetermined angle of rotation; if the subject is not in the predetermined angle of rotation, generating a correction control signal and transmitting the correction control signal to the actuating device to cause the person support surface to move; determining, based on the sensor data, whether the correction control signal moved the subject into the predetermined angle of rotation; and if the subject is in the predetermined angle of rotation, updating the database to indicate that the control signal and the correction control signal moved the person support surface into the predetermined angle of rotation.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a perspective view of an illustrative person support apparatus including a control system for lateral rotation of a person support surface, according to one or more embodiments shown and described herein;

FIG. 1B illustrates a side cross-sectional view of a portion of the person support surface of FIG. 1A, according to one or more embodiments shown and described herein;

FIG. 2 schematically depicts a block schematic diagram of an illustrative control system, according to one or more embodiments shown and described herein;

FIG. 3 schematically depicts an end view of the person support surface shown in FIG. 1A illustrating the rotation of the person support surface with a left side lifted, according to one or more embodiments shown and described herein;

FIG. 4 schematically depicts an end view of the person support surface shown in FIG. 1A illustrating the person support surface in a flat position, according to one or more embodiments shown and described herein;

FIG. 5 schematically depicts an end view of the person support surface shown in FIG. 1A illustrating the rotation of the person support surface with a right side lifted, according to one or more embodiments shown and described herein;

FIG. 6 illustrates a flow diagram of an illustrative method for controlling continuous lateral rotation therapy and automatically determining a positioning of a subject on a person support surface based on that positioning, according to one or more embodiments shown and described herein;

FIG. 7 illustrates a flow diagram of an illustrative method for controlling continuous lateral rotation therapy based on oxygen saturation of a subject on a person support surface, according to one or more embodiments shown and described herein;

FIG. 8A depicts a flow diagram of an illustrative method for controlling a person support apparatus, according to one or more embodiments shown and described herein; and

FIG. 8B depicts a continuation of the flow diagram of FIG. 8A.

### DETAILED DESCRIPTION

Embodiments described herein are directed to person support apparatuses and methods for optimizing subject movement during continuous lateral rotation therapy via machine learning. The person support apparatus utilizes a database of predetermined angles of rotation of continuous lateral rotation therapy routines to calculate control signals for moving the subject into the predetermined angle of rotation. The person support apparatus includes a sensor that monitors the subject and collects data about the subject, such that the person support apparatus may determine whether the subject has been moved into the predetermined angle of rotation based on the data collected by the sensor.

Various embodiments of the person support apparatus and methods for operation of the person support apparatus are described in more detail herein. Whenever possible, the same reference numerals will be used throughout the drawings to refer to the same or like parts.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

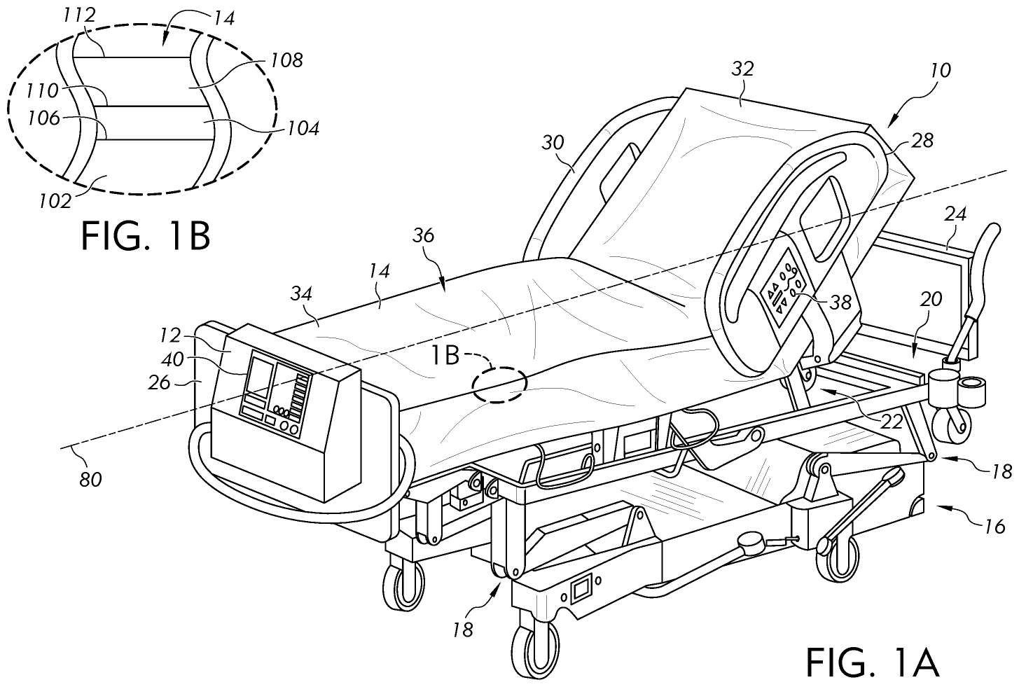

FIG. 1A illustrates a person support apparatus 10, (e.g., a bed) having a person support surface 14, (e.g., a mattress). A touch screen control system 12 including a user interface 40 is provided for controlling lateral rotation of the person support surface 14. The person support apparatus 10 includes a base frame 16, an intermediate frame 20 coupled to the base frame 16 by linkages 18, and an articulating deck frame 22 that is coupled to the intermediate frame 20 and that supports the person support surface 14. The person support apparatus 10 also includes a head end 24, a foot end 26, a left side rail 28, and a right side rail 30. A longitudinal axis 80 extends from the head end 24 to the foot end 26. The articulating deck frame 22 includes separate sections that articulate relative to the base frame 16 and relative to each other, for example, a mattress center section 36 that is height adjustable, and a mattress head section 32 and a mattress foot section 34 that are adjustable in elevation relative to the mattress center section 36. A control panel 38 is used to control articulation of the articulating deck frame 22. While the illustrated embodiment illustrates the left side rail 28 and the right side rail 30 extending along the mattress head section 32, either or both of the left side rail 28 and the right side rail 30 may also extend along either or both of the mattress center section 36 and the mattress foot section 34 in addition to or in lieu of extending along the mattress head section 32. In embodiments, rails (not illustrated) are provided at the head end 24 and/or the foot end 26 as illustrated with respect to the left side rail 28 and the right side rail 30.

The person support surface 14 includes a subject contact surface upon which the subject will rest. As described herein, the subject contact surface may be a top surface of the person support surface 14 which the subject contacts when they are placed on the person support surface 14. During use, however, top surface of the person support surface 14 may become contaminated from body fluids and microbiological substances. Such contamination may remain on the person support surface 14 and may go unrecognized unless the contamination is of sufficient size, which puts subsequent patients and staff at risk of exposure and possible infection from these contaminants.

Accordingly, in embodiments, this subject contact surface of the person support surface 14 may be configured to provide visual indication as to whether it has been properly cleaned, or that it needs to be cleaned, or to communicate that an incontinent event has occurred on the surface and that the subject needs attention. In embodiments, the subject contact surface is comprised in a cover that encases the person support surface 14. The subject contact surface (or a portion of the subject contact surface) may include hydro-chromic polymers, stimuli-responsive polymers, or smart polymers provided thereon or therein that induce a color change or change color when exposed to one or more extraneous materials. Extraneous materials may include, for example, water, cleaning products, bodily fluids, or various other materials or stimuli that may come in contact with the person support surface 14 and/or that may be utilized as part of a cleaning and disinfection protocol. The color or colorless induced signal may indicate sufficient contact between the person support surface 14 and the water, cleaning agents, or other stimuli needed for thorough cleaning and disinfection. After a period, the color or colorless induced signal will revert to the previous color or colorless state, indicating that the person support surface 14 is ready to be used by the next patient.

In embodiments, the subject contact surface (or a portion of the subject contact surface) may change from a color to colorless, or change from colorless to a color, or change from one color to another color when exposed to bodily fluids. The color change would indicate that areas of the subject contact need to be cleaned and/or that they have been cleaned. Once dry, the subject contact surface may return to its original state (i.e., original color or original colorless state). For example, the color change occurs when in contact with the bodily fluid but, once the fluid evaporates, the person support surface 14 will transition back to its original color/state.

The hydro-chromic polymers (or the smart polymers or the stimuli-responsive polymers) may be of various forms, such as inks, crystals, pastes, slurries, films, pigments, paints, dispersed into suspension mediums, etc., which are provided on the person support surface 14. The hydro-chromic polymers (or the smart polymers or the stimuli-responsive polymers) may be applied directly on of the subject contact surface of the person support surface 14 (e.g., on a textile of the subject contact surface that is knitted, woven, non-woven, a fiber, film, foam, or any combination thereof), or applied to a substrate that is provided on the subject contact surface, for example, as a film. In embodiments, the substrate may be a textile substrate that is knitted, woven, non-woven, a fiber, film, foam, or any combination thereof. The hydro-chromic polymers (or the smart polymers or the stimuli-responsive polymers) may provided on the subject contact surface of the person support surface 14 or on the substrate via coating, laminating, printing, finishing, or surface treatment. In embodiments, the hydro-chromic polymers are comprised in a waterproof polyurethane coating that is applied to the fabric to keep water and body fluids from entering the core of the person support surface 14. In embodiments, other materials compositions are mixed with the hydro-chromic polymers, such as a fire retardant agent. In some embodiments, the subject contact surface of the person support surface 14 includes a base color (or is coated with a material or film that provides it with the base color), and a hydro-chromic coating or lamination is provided over the subject contact surface color (or over the material coating or film that provides the base color). In these embodiments, the hydro-chromic coating or lamination has a second color different than the base color and, when provided over the subject contact surface (or over the material coating that provides the base color), covers (or obscures or masks) the base color; however, when the hydro-chromic coating or lamination is exposed to fluid (or becomes wet), the hydro-chromic coating or lamination becomes transparent such that the base color is visible through the hydro-chromic coating or lamination, thereby indicating that, for example, an incontinent event has occurred. In embodiments, the hydro-chromic coating or lamination returns to the second color, thereby once again covering or masking the base color, when it becomes dry.

Fluid ingress into the person support surface 14 will occur when the integrity of the person support surface 14 is compromised, thereby allowing blood and other fluids to penetrate the person support surface 14 and pose the risk of contamination and patient infection. Health care providers are responsible for product life-cycle management for products in the field that have reached the end of their ‘expected service life’ as defined in their instructions for use. Upon identification of end stages of the ‘expected service life,’ such as punctures, rips, tears, or other damage, the person support surface 14 may be removed from the field or rehabilitated back to its original, new state. However, fluid ingress into the person support surface 14 may occur through unnoticeable signs of wear before a complete failure of the person surface failure 14, such as a minor tear or abrasion.

Accordingly, the person support surface 14 may be configured to provide visual indication of wear, which will provide a means to more easily identify a specific degree of wear and thus proximity to an imminent state of fluid ingress. In embodiments, the composition of the person support surface 14 comprises an exterior color tinted polymer covering a textile substrate of the person support surface 14, and a penultimate layer of a differentiating color tinted polymer in between the exterior color tinted polymer and the textile substrate that is revealed through repeated abrasion of the person support surface 14 over time, commensurate to the ‘expected service life’ of the person support surface 14, wherein the differentiating color alerts the health care provider that the person support surface 14 is close to an imminent failure of fluid ingress and should be removed from the field.

The exterior color tinted polymer and the differentiating color tinted polymer may take various forms, such as pigment, ink, dye, paste, film, paint, or dispersed into suspension mediums. Also, the exterior color tinted polymer and the differentiating color tinted polymer may be applied to a textile substrate that is knitted, woven, non-woven, fiber, film, foam, or any combination thereof. Further, the exterior color tinted polymer and the differentiating color tinted polymer may be assembled on the textile substrate in a variety of manners, such as by stacking or layering the exterior color tinted polymer and the differentiating color tinted polymer by means of coating, laminating, printing, finishing, or surface treatment alone or in combination, with or without the coloration of the textile substrate.

In embodiments, the mattress ticking of the person support surface 14 is a polymer coated textile that comprises several layers of polymers over a textile base layer, wherein the person support surface 14 is utilizable or serviceable in the field to the extent that any one or more of the layers of polymer are present over the textile base layer. Each of the layers of polymer includes a different pigment (e.g., a pearlescent pigment) such that each of the layers comprises a different unique color that is different than a color of the textile base layer. As the top layer wears off, a different color of a layer beneath the top layer may be exposed, indicating that, while the top layer has been worn away, the layer beneath the top layer is still present and that the mattress ticking is still serviceable. This may allow the user to determine when to replace the mattress ticking, as color contrast may indicate the wear of the mattress for replacement. Thus, a user may determine the degree to which the exterior of the person support surface 14 has been worn or abraded and may determine whether it has been sufficiently worn such that it needs to be retired from service as evidenced via exposure of the textile base layer.

In embodiments, an adhesive layer is provided on the textile base layer, a pearlescent pigment layer is provided over the adhesive layer, and then a polymer layer is provided over the pearlescent pigment layer such that the pearlescent pigment layer is not visible through the polymer layer. In some embodiments, the polymer layer comprises two or more polymer layers that are provided over the pearlescent pigment to ensure that the pearlescent pigment layer is not visible through the polymer layers. With these arrangements, a color of the pearlescent pigment layer becomes visible after the polymer layer(s) have been sufficiently abraded (or worn away) to expose a color of the pearlescent pigment layer underlying the polymer layer(s), which thereby indicates that the mattress ticking needs replacement and/or maintenance.

As exemplified in FIB. 1B, the person support surface 14 may comprise a textile base layer 102 (e.g., a Nylon ticking) acting as a substrate, a middle polymer layer 104 provided on a surface 106 of the textile base layer 102, and a top polymer layer 108 provided on a surface 110 of middle polymer layer 104. The middle polymer layer 104 is mixed with a pigment such that it has a first color and the top polymer layer 108 is mixed with a different pigment such that it has a second color that is different from the first. Also, the first color and the second color of the middle polymer layer 104 and the top polymer layer 108, respectively, may each be different than a color of the textile base layer 102. The top polymer layer 108 is sufficiently thick such that the first color of the middle polymer layer 104 is not visible through the top polymer layer 108. As the top polymer layer 108 is worn and abraded over time, the middle polymer layer 104 becomes exposed such that the first color thereof is visible. The middle polymer layer 104 is sufficiently thick such that a color of the textile base layer 102 is not visible through the middle polymer layer 104.

During use, a top surface 112 of the top polymer layer 108 is exposed and may be in contact with external objects (e.g., the subject, medical devices, etc.), such that its color (i.e., the second color) is visible and subject to friction from such external objects. After being abraded (or worn away) by friction by use over time, the top polymer layer 108 wears off and the middle polymer layer 104 underlying the top polymer layer 108 becomes exposed such that the first color thereof is revealed, which provides indication that the person support surface 14 has experienced at least some degree of wear. Exposure of the first color of the middle polymer layer 104 may be indicative of various states (or quantities) of wear, for example, it may indicate that the person support surface 14 is still serviceable or utilizable in the field but has a certain amount of use left. Then, after further use, the middle polymer layer 104 becomes abraded (or worn away) by friction such that the textile base layer 102 becomes exposed, which provides indication that the person support surface 14 is overly worn and its ticking needs replacement or maintenance. In embodiments, the middle polymer layer 104 the top polymer layer 108 are provided as a film, and the film is provided on the person support surface 14. In embodiments, the middle polymer layer 104 comprises a plurality of middle polymer layers, where each of the middle polymer layers has a different color or appearance that provides an indication of current wear and/or remaining life of the person support surface 14. In these embodiments, abrasion of the top polymer layer 108 will expose an upper-most middle polymer layer which indicates a certain amount of wear (and/or a certain amount of remaining life) of the person support surface 14), and then abrasion of the upper-most middle polymer layer will expose a next upper-most middle polymer layer which indicates a certain amount of additional wear (and/or a certain amount of less remaining life) of the person support surface 14, and so on. The film may be manufactured by laying down each layer, one at a time, and then the film may be applied to the person support surface 14.

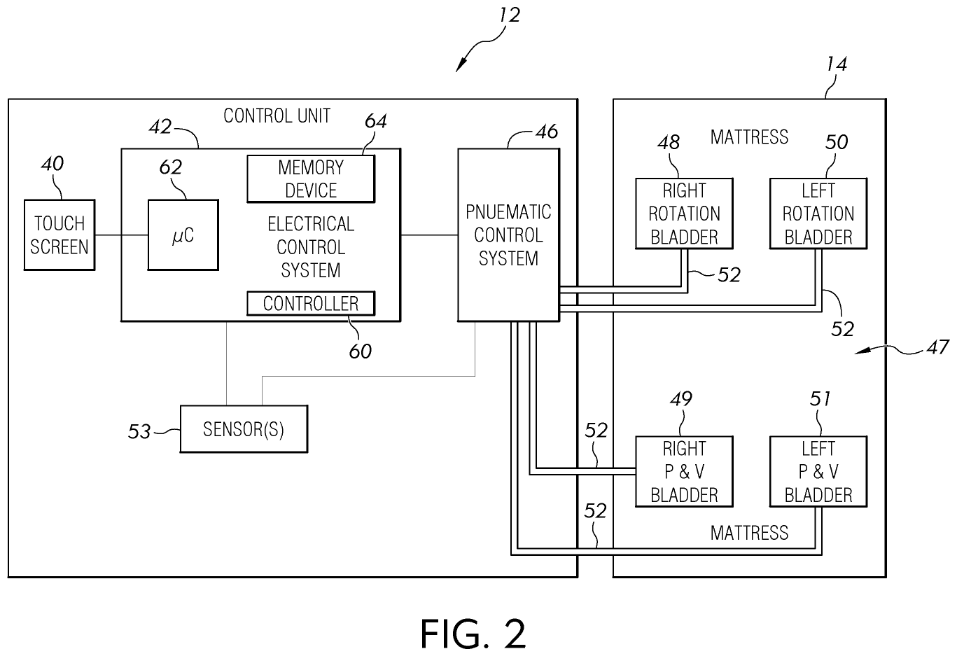

FIG. 2 illustrates the control system 12 and its interaction with the person support surface 14. The control system 12 includes the user interface 40, an electrical control system 42, and a pneumatic control system 46. The electrical control system 42 includes a controller 60, a microprocessor 62, and a memory device 64. The memory device 64 may store instructions that, when executed by the microprocessor 62, perform various operations as described herein. The electrical control system 42 is electrically coupled to the user interface 40 and the pneumatic control system 46. The memory device 64 includes instructions that, when carried out by the microprocessor 62 cause the controller 60 to control the electrical control system 42 and the pneumatic control system 46.

The person support surface 14 includes an actuating device, for example, a bladder system 47 having a right rotation bladder 48 and a left rotation bladder 50 that provides positioning and/or therapy, for example, continuous lateral rotation therapy, percussion and/or vibration therapy, as discussed in more detail herein, and positioning to assist turning of the subject. In embodiments, the bladder system 47 may also include a right P&V (percussion and vibration) bladder 49 and a left P&V bladder 51. As such, the right rotation bladder 48 and a left rotation bladder 50 may be specifically utilized for facilitating rotation of the person support surface 14, while the right P&V bladder 49 and the left P&V bladder 51 simultaneously provide percussion and/or vibration without affecting rotation of the person support surface 14. It should be appreciated that the right P&V bladder 49 and the left P&V bladder 51 may include the same structure as the right rotation bladder 48 and the left rotation bladder 50.

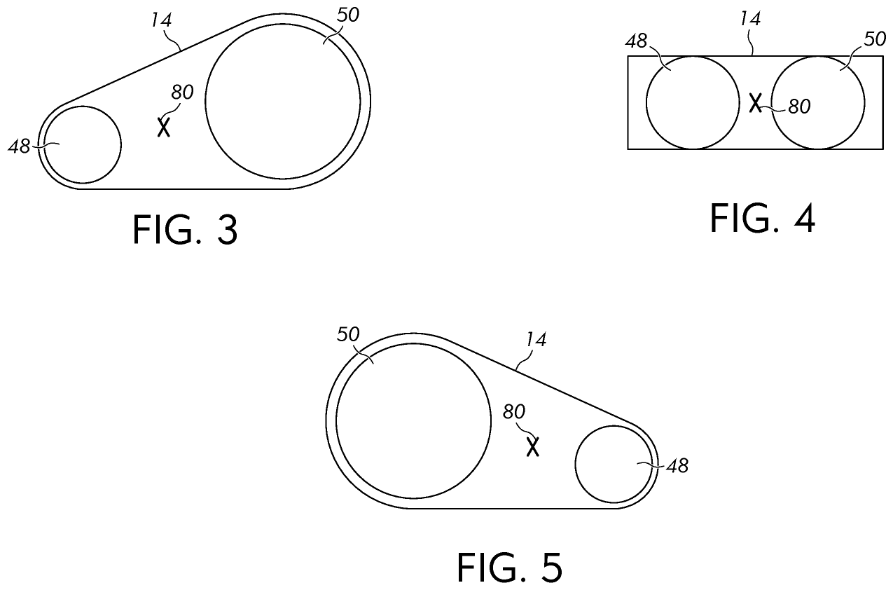

The bladder system 47, the right rotation bladder 48, and the left rotation bladder 50 are obscured from view in FIG. 1A, but are schematically illustrated relative to the person support surface 14 in FIGS. 3-5. As shown in FIGS. 3-5, the right rotation bladder 48 and the left rotation bladder 50 may be provided within the person support surface 14. For example, where the person support surface 14 is a mattress, the right rotation bladder 48 and the left rotation bladder 50 may be provided underneath the textile base layer 102 of the mattress ticking. In some embodiments, one or more layers or padding (e.g., foam layers) are provided beneath the textile base layer 102 of the mattress ticking, and the right rotation bladder 48 and the left rotation bladder 50 may be provided beneath such one or more layers or padding. In embodiments, the person support surface 14 includes a base plate proximate positioned at a bottom side of the person support surface 14 (opposite to the subject contact surface upon which the subject will rest), proximate to the articulating deck frame 22, and the right rotation bladder 48 and the left rotation bladder 50 may be positioned on top of the base plate such that they are positioned in between the base plate and the subject contact surface of the person support surface 14. In these embodiments, a top plate may be provided over the right rotation bladder 48 and the left rotation bladder 50, such that the right rotation bladder 48 and the left rotation bladder 50 are provided in between the top plate and the base plate. In such embodiments, inflation or deflation of the right rotation bladder 48 and the left rotation bladder 50 will cause the top plate to move relative to the base plate so as to orient the subject contact surface at an angle. In embodiments, the right rotation bladder 48 and the left rotation bladder 50 each comprise an individual bladder. In other embodiments, the right rotation bladder 48 and/or the left rotation bladder 50 each comprise a plurality of bladders. It should be appreciated that the top plate and/or base plate are optional and that, in some embodiments, turning is accomplished via bladders, such as the bladder system 47 that includes the right rotation bladder 48 and the left rotation bladder 50.

In embodiments, the right rotation bladder 48 and the left rotation bladder 50 each comprise an outer enclosure of urethane coated nylon which provides an air impermeable enclosure. In embodiments, a two layered foam structure is provided inside of each enclosure, wherein such two layered foam structure includes an upper layer and a lower layer, and these layers are glued together. The foam structure is deformable under load, but resiliently expands to fill the interior space of the enclosure, even when the enclosures are inflated beyond their default or resting state. The enclosures may each include a pressure relief or check valve.

The control system 12 controls the right rotation bladder 48, the left rotation bladder 50, the right P&V bladder 49, and the left P&V bladder 51 by receiving user inputs at the user interface 40. The electrical control system 42 controls and monitors the pneumatic control system 46 based on the user inputs. The user interface 40 provides a graphical interface for setting and monitoring the electrical control system 42 and the pneumatic control system 46, which in turn actuates pneumatic aspects of the right rotation bladder 48, the left rotation bladder 50, the right P&V bladder 49, and the left P&V bladder 51. The pneumatic control system 46 is coupled to the right rotation bladder 48, the left rotation bladder 50, the right P&V bladder 49, and the left P&V bladder 51 by pneumatic lines 52. In embodiments, one or more of the pneumatic lines 52 may be embodied in a manifold. In embodiments, the user interface 40 comprises one or more left rotation buttons for entering parameters associated with the left rotation bladder 50 and/or the left P&V bladder 51 and one or more right rotation buttons for entering parameters associated with the right rotation bladder 48 and/or the right P&V bladder 49.

The pneumatic control system 46 may also include a fluid source coupled to each of the right rotation bladder 48 and the left rotation bladder 50 for inflating or deflating of the right rotation bladder 48 and the left rotation bladder 50. Thus, the fluid source may comprise a pump and/or a fluid reservoir. The pump may direct fluid into and out of the right rotation bladder 48 and the left rotation bladder 50. In embodiments, the fluid pumped into the right rotation bladder 48 and the left rotation bladder 50 is air that the pump draws from the ambient environment, but in other embodiments, the pump draws a fluid from the fluid reservoir and pumps that fluid into the right rotation bladder 48 and the left rotation bladder 50. In this manner, the fluid source is configured to provide fluid to the right rotation bladder 48 and/or the left rotation bladder 50 to thereby fill the right rotation bladder 48 and/or the left rotation bladder 50 and cause the top plate to move relative to the base plate, such that the movable section of the person support surface 14 may be angled or elevated. In embodiments, the right rotation bladder 48 and the left rotation bladder 50 are each connected to a manifold of the bladder system 47, and the fluid source is also coupled to the manifold for inflating or deflating the right rotation bladder 48 and/or the left rotation bladder 50. As hereinafter described, pressure sensors (e.g., piezoelectric pressure sensors) may be operatively coupled to each of the right rotation bladder 48 and the left rotation bladder 50 for measuring the pressure within therein. This pressure may be utilized by the control system 12 to determine an amount of inflation (or deflation) of the right rotation bladder 48 and the left rotation bladder 50.

The control system 12 includes one or more sensors 53 communicably coupled to the electrical control system 42 and/or the pneumatic control system 46 for detecting one or more parameters. The one or more sensors 53 monitor operation of the pneumatic control system 46 and/or the bladders 48, 49, 50, 51, and also monitors the subject supported on the person support apparatus 10 as they undergo rotation therapy via to inflation and deflation of the bladders 48, 49, 50, 51. Thus, the one or more sensors 53 may provide feedback control of the bladders 48, 49, 50, 51 to the pneumatic control system 46 and/or to the electrical control system 42. The one or more sensors 53 may also provide feedback to the pneumatic control system 46 and/or to the electrical control system 42 that is indicative of how the subject is responding to the rotation therapy. For example, the one or more sensors 53 may provide feedback as to how the person support apparatus 10 is providing rotation therapy based on the actual turning, rotation, or position of the subject as compared to the rotation therapy program set by the user input. While the one or more sensors 53 are schematically depicted in FIG. 2 as being part of the control system 12, any one or more of the one or more sensors 53 may be provided elsewhere. For example, one or more of the one or more sensors 53 may be provided in or on the person support surface 14, on the frame of the person support apparatus 10, on the subject, etc.

The one or more sensors 53 may include one or more sensors positioned about the person support apparatus 10. In embodiments, the one or more sensors 53 include one or more sensors positioned on the left side rail 28 and/or the right side rail 30. In embodiments, the one or more sensors 53 include one or more sensors positioned at the head end 24 (e.g., at a headboard) and/or at the foot end 26 (e.g., at a footboard) of the person support apparatus 10. In embodiments, the one or more sensors include sensors positioned at the head end 24, the foot end 26, the left side rail 28, and the right side rail 30.

In embodiments, the one or more sensors 53 include one or more sensors positioned in the mattress, for example, in the mattress head section 32, the mattress center section 36, and/or the mattress foot section 34. In embodiments, the one or more sensors 53 include one or more sensors positioned in the intermediate frame 20 and/or the base frame 16 of the person support apparatus 10. In embodiments, the one or more sensors 53 include one or more sensors positioned on the subject who is supported on the person support apparatus 10, for example, in the form of a wearable sensor device.

The one or more sensors 53 may include various types of sensors or combinations of sensors. In embodiments, the one or more sensors 53 may include pressure sensors, capacitive sensors, inductive sensors, optical sensors, infrared sensors, load beams, load cells, cameras, video cameras, moisture sensors, temperature sensors, etc.

In embodiments, the one or more sensors 53 may measure weight and/or height of the subject. For example, the one or more sensors 53 may include weight sensors and optical sensors for monitoring a subject's height, and data from the one or more sensors 53 may be utilized to calculate a weight distribution of the subject based on the subject's measured weight and height, which may in turn be utilized to improve consistency of turning across various subjects sharing or having similar weight distribution.

In embodiments, the one or more sensors 53 may include sensors configured to measure or monitor physiological conditions or biomarkers of the subject, for example, such as oxygen saturation (SpO2), skin degradation, heart rate, blood pressure, etc. In embodiments, the one or more sensors 53 include wearable sensors, such as transdermal wearable sensors, optical wearable sensors, and mechanical wearable sensors. Examples of such mechanical wearable sensors include piezoelectric sensors, piezoresistive sensors, piezocapacitive sensors, and triboelectric sensors. With regard to skin degradation, a sensor may be provided that measures moisture on the subject' skin. Also with regard to skin degradation, a sensor may be provided that measures or monitors skin color, swelling, temperature, and/or damage. For example, a sensor for measuring/monitoring skin degradation may include a thermal camera that captures changes in skin temperature over a particular time period (e.g. over the course of the subject's stay in a medical facility). Such a thermal camera could be installed such that it continuously monitors the subject, or such thermal camera could be utilized by the caregiver to actively take an image of an area of skin or suspected injury on as needed or as desired basis, or such thermal camera may be configured to capture video at certain times, such as passive video or image capture during sheet changes.

Also, the one or more sensors 53 may comprise pressure sensors that monitor pressure experienced at an area of the subject's skin. For example, a pressure sensor may be provided in a bandage or dressing that is applied to an area of the subject, and the pressure sensor in the bandage or dressing may monitor pressure experienced at the particular area of the subject's body. As hereinafter described, data from such pressure sensor may be utilized to monitor pressure off-loading.

Where the one or more sensors 53 includes load beams and/or load cells, such load beams and/or load cells may be configured to detect a pressure on the person support surface 14. In various cases, the load cell(s) and/or load beam(s) can include one or more strain gauges, one or more piezoelectric load cells, a capacitive load cell, an optical load cell, any device configured to output a signal indicative of an amount of pressure applied to the device, or a combination thereof. For example, the load cell(s) and/or load beam(s) may detect a pressure (e.g., weight) of the subject on the person support surface 14. In some cases, the person support apparatus 10 includes multiple load cells and/or load beams that respectively detect different pressures on the person support surface 14 in different positions along the person support surface 14. In some instances, the person support apparatus 10 includes four load cells arranged at four corners of the subject contact surface of the person support surface 14, which respectively measure the pressure of the subject on the person support surface 14 at the four corners of the person support surface 14. In embodiments, the person support apparatus 10 includes one or more load beams longitudinally extending between the head end 24 and the foot end 26 of the person support surface 14.

The one or more sensors 53 of the person support apparatus 10 may comprise one or moisture sensors. The moisture sensor(s) may be configured to measure a moisture on a surface (e.g., the resting surface) of the person support surface 14. For example, the moisture sensor(s) can include one or more capacitance sensors, one or more resistance sensors, one or more thermal conduction sensors, or a combination thereof. In some cases, the moisture sensor(s) include one or more fiber sheets configured to propagate moisture to the moisture sensor(s). In some cases, the moisture sensor(s) can detect the presence or absence of moisture (e.g., sweat or other bodily fluids) disposed between the person support surface 14 and the subject resting thereon. These moisture sensors may be utilized together with, or in lieu of, the above described hydro-chromic polymers.

In various examples, the one or more sensors 53 of the person support apparatus 10 can include one or more temperature sensors. The temperature sensor(s) may be configured to detect a temperature of the subject and/or the person support surface 14. In some cases, the temperature sensor(s) includes one or more thermistors, one or more thermocouples, one or more resistance thermometers, one or more Peltier sensors, or a combination thereof.

In embodiments, the one or more sensors 53 of the person support apparatus 10 can include one or more cameras. The camera(s) may be configured to capture images of the subject, the person support surface 14, or a combination thereof. In various cases, the camera(s) may include radar sensors, infrared cameras, visible light cameras, depth-sensing cameras, or any combination thereof. In some examples, infrared images may indicate, for instance, a temperature profile of the subject and/or the person support surface 14. Thus, the camera(s) may be a type of temperature sensor. In addition, the images may indicate a position or orientation of the subject and/or the person support surface 14, even in low-visible-light conditions. For example, the infrared images may capture a position of the subject during a night environment without ambient lighting in the vicinity of the subject and/or the s person support surface 14. In some cases, the camera(s) may include one or more infrared video cameras. The camera(s) may include at least one depth-sensing camera configured to generate a volumetric image of the subject, the person support surface 14, and the ambient environment. According to various implementations, the images and/or videos captured by the camera(s) are indicative of a position and/or a movement of the subject over time. Also, the camera(s) may be utilized to measure a height of the subject.

According to some examples, the one or more sensors 53 of the person support apparatus 10 can include one or more video cameras. The video camera(s) may be configured to capture videos of the subject and the person support surface 14. The videos may include multiple images of the subject and/or the person support surface 14. Thus, the videos captured by the video camera(s) may be indicative of a position and/or movement of the subject over time. In some examples, the video camera(s) capture visible light videos, changes in radar signals over time, infrared videos, or any combination thereof.

Where utilized, the cameras and/or video cameras may be coupled to either or both of the side rails 28, 30 and/or to either or both of the head end 24 and/or the foot end 26. In embodiments, the cameras and/or video cameras are mounted on an extension (e.g., a metal structure) attached to the head end 24, the foot end 26, the left side rail 28, and/or the right side rail 30. In some embodiments, the camera(s) and/or video camera(s) are attached to a wall and/or ceiling of the room containing the person support apparatus 10. In some embodiments, the camera(s) and/or video camera(s) are attached to a cart (e.g., a support structure on wheels) that is located in the vicinity of the person support apparatus 10.

In various cases, the one or more sensors 53 of the person support apparatus 10 are configured to monitor one or more parameters of the subject and to generate sensor data associated with the subject. In various cases, the one or more sensors 53 convert analog signals (e.g., pressure, moisture, temperature, light, electric signals, or any combination thereof) into digital data that is indicative of one or more parameters of the subject. As used herein, the terms “parameter,” “subject parameter,” and their equivalents, can refer to a condition of the subject and/or their surrounding environment. In this disclosure, a parameter of the subject can refer to a position of the subject, a movement of the subject over time (e.g., rotation of the subject into a left or right rotation position), a pressure between the subject and a surface of the person support surface 14, a moisture level between the subject and the person support surface 14, a temperature of the subject, a vital sign of the subject (e.g., a blood pressure, a peripheral oxygenation level, a heart rate, a respiration, a blood volume, or some other vital sign), a nutrition level of the subject, a medication administered and/or prescribed to the subject (e.g., a vasopressor or any other medication that impacts pressure injury risk), a previous condition of the subject (e.g., the subject was monitored in an ICU, in dialysis, presented in an emergency department waiting room, etc.), a skin tone of the subject (e.g., which may impact whether care providers readily identify pressure injuries on the skin of the subject), circulation of the subject (e.g., restricted blood flow), a pain level of the subject, the presence of implantable or semi-implantable devices (e.g., ports, tubes, catheters, other devices, etc.) in contact with the subject, or any combination thereof. Optionally, a parameter is specific to a limb, such as an arm or a leg of the subject. In various examples, the person support apparatus 10 generates data indicative of one or more parameters of the subject using the load cell(s), the moisture sensor(s), the temperature sensor(s), the cameras, the video camera(s), or a combination thereof. The data generated by the person support apparatus 10 be referred to herein as “sensor data.”

The person support apparatus 10 may further include a transmitter, for example, in the control system 12. The transmitter may be configured to transmit the sensor data from the person support apparatus 10 over one or more communication networks. The communication network(s) may include one or more wired networks (e.g., at least one wired local area network (LAN), at least one fiber-optic network, or some other type of wired network), one or more wireless networks (e.g., at least one wireless LAN, at least one WI-FI network, at least one BLUETOOTH network, at least one cellular network, or some other type of wireless network), or a combination thereof. In some cases, the communication network(s) include one or more wide area networks (WANs), such as the Internet. In some cases, the transmitter includes a transceiver configured to receive signals from the communication network(s).

In embodiments, the person support apparatus 10 may transmit the sensor data to an external identification system and/or an external electronic medical record (“EMR”) system over the communication network(s). The external identification and/or EMR system may be configured to transmit and/or receive data over the communication network(s). The external identification and/or EMR system may be embodied in hardware, software, or a combination thereof. In some cases, the external identification and/or EMR system is hosted on one or more computing devices located in a local environment of the person support apparatus 10. For example, the external identification and/or EMR system may be hosted on an on-premises server located in a hospital in which the person support apparatus 10 is also located. In some cases, the external identification and/or EMR system is hosted by one or more computing devices located remotely from the premises of the person support apparatus 10. For example, the external identification and/or EMR system may be hosted in a distributed computing environment, such as a cloud computing environment. As described herein, the control system 12 employs a computing model for controlling the pneumatic control system 46, and the external identification system may be configured to generate, manage, and/or adjust the computing model based on the sensor data and/or EMR data stored by the external EMR system.

The pneumatic control system 46 generally provides pressure or vacuum to, or sealing of, the pneumatic lines 52 to selectively inflate, deflate, or maintain the inflation of the right rotation bladder 48 and the left rotation bladder 50. For example, to rotate a subject on the person support surface 14 toward the right relative to a longitudinal axis 80 of the person support surface 14, i.e., into a right rotation position, the pneumatic control system 46 actuates the right rotation bladder 48 located under the subject's right side to a volume or capacity that is less than a volume or capacity of the left rotation bladder 50 located under the subject's left side, as illustrated in FIG. 3. In another example, to rotate a subject on the person support surface 14 toward the left relative to the longitudinal axis 80, i.e., into a left rotation position, the pneumatic control system 46 actuates the left rotation bladder 50 located under the subject's left side to a volume or capacity that is less than a volume or capacity of the right rotation bladder 48 located under the subject's right side, as illustrated in FIG. 5. To position the subject in a flat orientation, as shown in FIG. 4, the left rotation bladder 50 and the right rotation bladder 48 are actuated to substantially the same volume or capacity.

Alternatively, only one of or a portion of the right rotation bladder 48 and the left rotation bladder 50 of the bladder system 47 may be actuated by inflation or deflation to laterally rotate a subject on the person support surface 14 to different rotational positions. Additionally, the bladder system 47 may comprise bladders that are associated with the person support surface 14 in an arrangement different from that shown in FIGS. 2-5, as is known in the art. For example, the bladder system 47 may include vertically stacked bladders, one of which is normally inflated and another that is inflated or deflated to provide rotation. Additionally, the pneumatic control system 46 may also provide other pneumatic control, for example, to provide percussion, vibration, or other desired person therapy or positioning, as discussed in more detail herein. The pneumatic control system 46 and the bladder system 47 of the person support surface 14 may alternatively include a non-pneumatic mechanical or electrometrical control and motion system, or a pneumatic system utilizing movement elements other than bladders. Regardless of the configuration and operation of the right rotation bladder 48 and the left rotation bladder 50 of the bladder system 47, the control system 12 may be configured to provide the caregiver with a preview of the various rotation positions to be implemented on the subject during a particular routine of continuous lateral rotation therapy, to thereby enable a caregiver to quickly assess whether any IV lines, tubes, or the like will be occluded by rotating the subject to the predetermined angles of rotation without dwell or hold time

During continuous lateral rotation therapy, the subject is rotated, from their flat orientation, into the right rotation position or into the left rotation position. In particular, the subject will be rotated into the right rotation position at a predetermined angle of rotation on their right side for a rotation period of time, the subject will be rotated into the left rotation position at a predetermined angle of rotation on their left side for a rotation period of time. The subject may also be held in the flat orientation for a period of time. For example, the subject may be rotated to 40 degrees to each side for 10 minutes and flat for 10 minutes. Generally, the right rotation bladder 48 and the left rotation bladder 50 are actuated over a period of dwell or hold time that enables the subject to slowly be moved into the predetermined angle of rotation. It will be appreciated that any rotational angle may be desired for any period of time depending on the particular continuous lateral rotation therapy routine. The angle of rotation and/or the duration of time at which the subject is held at such angle of rotation may depend on various physical characteristics of the subject, such as their height and/or weight. For example, the angle of rotation and/or the duration of time may depend on a weight distribution of the subject. Also, the angle of rotation and/or the duration of time at which the subject is held at such angle of rotation may depend on various physiological characteristics of the subject, such as the subject's SpO2 and/or other vital signs of the subject.

The memory device 64 may store various pre-programmed continuous lateral rotation therapy routines that, when selected at the user interface 40 by the caregiver, cause the pneumatic control system 46 to control the bladder system 47 to inflate and deflate the right rotation bladder 48 and the left rotation bladder 50 according to the pre-programmed routine, thereby subjecting the subject to the pre-programmed continuous lateral rotation therapy routine. The pre-programmed continuous lateral rotation therapy routines may be stored in a database. Also or instead, the caregiver may manually enter, via the user interface 40, a user input into the database that is indicative of a particular continuous lateral rotation therapy routine that the caregiver would like the subject to undergo, such that the subject experiences that particular continuous lateral rotation therapy according to the user input stored in the database. Accordingly, the control system 12 will be able to reference, whether from a pre-programmed routine or previously entered user input, a predetermined turn angle that the subject should experience as they undergo any particular continuous lateral rotation therapy routine.

The control system 12 is programmed to determine how much to inflate or deflate the right rotation bladder 48 and the left rotation bladder 50 to orient the subject at the predetermined turn angle that the subject should experience as they undergo any particular continuous lateral rotation therapy routine. For each predetermined turn angle, the database has a corresponding predetermined control signal for inflating/deflating the right rotation bladder 48 and the left rotation bladder 50 to orient the subject at the predetermined turn angle.

Multiple continuous lateral rotation therapy routines may be conducted, on the same subject and/or different subjects, and data about how the subject responds to the particular control signals of each of the routines may be compiled and added into database and stored in the memory device 64 such that the control system 12 may determine how much to inflate or deflate the right rotation bladder 48 and the left rotation bladder 50 to achieve a particular predetermined angle of rotation or orientation of the subject. For example, where a particular continuous lateral rotation therapy routine is conducted on a particular subject (or on a subject having certain characteristics), whether or not a control signal does results in the subject being moved into the predetermined turn angle may be stored in the database. If the control signal does not cause positioning of the subject into the predetermined turn angle, the control system 12 may correct positioning of the subject by generating a new control signal that will move the subject into the predetermined turn angle, and that new control signal may be stored in the database.

Thus, the database will grow over time as more and more continuous lateral rotation therapy routines are performed and data gathered therefrom is added to the database, such that the control system 12 will more accurately control inflation or deflation of the right rotation bladder 48 and the left rotation bladder 50 to achieve the predetermined angle of rotation or orientation of the subject during a particular routine. Thus, using the turn data database, an algorithm for controlling inflation or deflation of the right rotation bladder 48 and the left rotation bladder 50 may be developed over time. The algorithm may control bladder pressure based on multiple inputs (e.g., at least weight) and may be a machine learning or the like that learns from the plurality of inputs to become more accurate relative to the clinical need over time. Also, the data in the turn data database may be indexed, for example, by particular subject, by one or more subject characteristics, etc., such that the generated algorithm is generated from data collected from that particular subject and/or data associated with characteristics of the particular subject.

The database may comprise various types of data, such as performance data that is indicative how well the subject is moved into the predetermined angle of rotation. The one or more sensors 53 are positioned to monitor the subject and generate the performance data, which is transmitted to the control system 12 and utilized by the control system 12 to optimize continuous lateral rotation therapy routines. The performance data generated by the one or more sensors 53 may comprise actual rotation angle experienced by the subject, distance of the subject to either or both of the side rails 28,30 (which would indicate lateral shifting of the subject), distance of the subject to either or both of the head end 24 and/or the foot end 26 (which would be indicative of longitudinal shifting of the subject), pressure offloading at one or more areas on the subject's body, etc. In embodiments, the database may include physiological data or biomarkers of the subject, for example, such as SpO2, skin degradation, heart rate, blood pressure, etc., such that the algorithm may be at least partially derived from such data or biomarkers, and the control system 12 may utilize such physiological data or biomarkers, with or without performance data regarding how well the subject is moved into the predetermined angle of rotation, to optimize continuous lateral rotation therapy.

The database may be specific to a particular subject or, as mentioned above, the data in the database may be indexed by each particular subject. For example, multiple continuous lateral rotation therapy routines may be conducted on a particular subject and data about how that subject responds to the inputs of those routines may be compiled into a subject specific database that is stored in the memory device 64 for reference by the control system 12, such that the control system 12 will be able to determine how much to inflate or deflate the right rotation bladder 48 and the left rotation bladder 50 to achieve a particular predetermined angle of rotation or orientation of that particular subject. In this example, the database stored on the memory device 64 may contain a plurality of subject specific databases for each of the various subjects that may be referenced when the particular subject undergoes a continuous lateral rotation therapy routine. Using data within the subject specific database, a subject specific algorithm for controlling inflation or deflation of the right rotation bladder 48 and the left rotation bladder 50 may be developed over time. In this embodiment, the control system 12 may recognize that the particular subject is undergoing a continuous lateral rotation therapy routine based on input from the caregiver indicating the same, or the control system 12 may automatically recognize that the particular is undergoing a continuous lateral rotation therapy routine via the one or more sensors 53. For example, the one or more sensors 53 may include cameras and, using facial recognition software stored in the memory device 64, the control system 12 identify the particular subject and retrieve their particular subject specific database and their particular subject specific algorithm. If the control system 12 does not recognize the particular subject, a new subject specific database may be created for that particular subject such that a new subject specific algorithm for controlling inflation or deflation of the right rotation bladder 48 and the left rotation bladder 50 may be developed over time for that particular new subject. In embodiments, the control system 12 may communicate with the external EMR system, which contains subjects' EMRs, and utilize data from the EMR system to identify the particular subject.

The database may be specific to one or more shared characteristics of subjects or, as mentioned above, the data in the database may be indexed by one or more shared characteristics. The shared characteristics may include physical characteristics, for example, height and/or weight, such that subjects having similar heights and/or weights (or within the same height range and/or weight range) may be considered to have one or more shared characteristics. However, the shared characteristics may also or instead include physiological characteristics such as, for example, SpO2, such that subjects having the same SpO2 (or being within the same SpO2 range), may be considered to have one or more shared characteristics. In this manner, data from subjects sharing one or more characteristics may be indexed together based on the shared characteristic(s) or may be compiled together into a shared characteristic database that is stored in the memory device 64 for reference by the control system 12, such that the control system 12 will be able to determine how much to inflate or deflate the right rotation bladder 48 and the left rotation bladder 50 to achieve a particular predetermined angle of rotation or orientation for a subject having the one or more shared characteristics. In embodiments, the indexing or the shared characteristic database may be based on subjects having a certain height (or height range), a certain weight (or weight range), and/or a certain center or gravity (or center of gravity range), etc. Using data indexed by shared characteristic(s) or data within the shared characteristic database, a characteristic specific algorithm for controlling inflation or deflation of the right rotation bladder 48 and the left rotation bladder 50 may be developed over time. In this embodiment, the control system 12 may recognize that the subject having one or more particular shared characteristics is undergoing a continuous lateral rotation therapy routine based on input from the caregiver indicating the same, or the control system 12 may automatically recognize that the subject undergoing a continuous lateral rotation therapy routine has one or more particular shared characteristics via the one or more sensors 53. For example, the one or more sensors 53 may include weight sensors to measure the subject's weight and/or sensors to measure the subject's height, and/or the control system 12 may be programmed to determine the subject's center of gravity utilizing data collected from the one or more sensors 53.

The database may also include data from the EMR and such EMR data may be utilized to build or modify the algorithm. For example, medical record data may include information about whether a subject developed a pressure injury and/or had an adverse respiratory or pulmonary response. Here, the control system 12 may be configured to access subjects' EMRs and automatically update the database with information about the subjects who develop pressure injuries during continuous lateral rotation therapy routines or the caregiver may manually log that information into the database. Not only may the database may pull information from the subject's EMIR regarding their skin health, but the database may be pull information regarding the subject's pulmonary condition progression and/or respiratory condition progression during continuous lateral therapy. The occurrence of these pressure injuries and/or adverse pulmonary/respiratory responses may be correlated with the angle of rotation experienced by the subject, for example, via the one or more sensors 53 which may be positioned on the side rails 28, 30 of the bed. Using this information, the control system 12 may determine whether certain subjects or whether subjects having certain shared characteristics suffered pressure injuries or had adverse pulmonary/respiratory responses following their continuous lateral rotation therapy routines, and then the control system 12 may utilize that information to improve the algorithm in an attempt to lessen the likelihood of subjects developing such pressure injuries or adverse pulmonary/respiratory responses in response to continuous lateral rotation therapy. This may also be useful in a clinical setting, as both the degree to which a subject is turned and their response to said therapy could both be recorded. In addition, this data could be used to determine which subjects actually benefit from continuous lateral rotation therapy and in what amounts/degree of turn/etc. could be used to further develop the algorithm.

In embodiments, the control system 12 optimizes continuous lateral rotation therapy by comparing an actual orientation or position experienced by the subject to the predetermined angle of rotation for the particular continuous lateral rotation therapy routine. Using the algorithm, the control system 12 calculates a control signal to inflate and/or deflate the right rotation bladder 48 and/or the left rotation bladder 50 an amount sufficient to move the subject into the predetermined angle of rotation as specified in the continuous lateral rotation therapy routine, regardless of whether the routine is a pre-programmed routine or a user-input routine. As the subject undergoes any such continuous lateral rotation therapy routine, the control may utilize the one or more sensors 53 to monitor the subject and determine whether the subject has actually been turned or positioned into the predetermined angle of rotation as specified in the continuous lateral rotation therapy routine. If the control system 12 determines that the actual angle of rotation experienced by the subject equals the predetermined angle of rotation set for the continuous lateral rotation therapy routine, for example, by comparison, the control system 12 updates the database to indicate the same and continues with the continuous lateral rotation therapy routine.

If the control system 12 determines that the actual angle of rotation experienced by the subject is not equal to the predetermined angle of rotation set for the continuous lateral rotation therapy routine, for example, by comparison, the control system 12 updates the database to indicate that the control signal did not result in the predetermined angle of rotation, and then the control system 12 utilizes the algorithm to calculate a new control signal to further inflate and/or deflate the right rotation bladder 48 and/or the left rotation bladder 50 an amount sufficient to move the subject into the predetermined angle of rotation as specified in the continuous lateral rotation therapy routine, during which the control system 12 monitors the subject via the one or more sensors 53. If the control system 12 determines that the new control signal resulted in rotation of the subject into the predetermined angle of rotation set for the continuous lateral rotation therapy routine, for example, via comparison, the control system 12 updates the database to indicate the same and continues with the continuous lateral rotation therapy routine. For example, if a set point (i.e., the predetermined angle of rotation) is X degrees and any of the one or more sensors 53 in the side rails 28, 30 detect that the subject is actually turned to X+5 degrees, air will be let out of the right rotation bladder 48 and/or the left rotation bladder 50 until the subject reaches the set point (i.e., the predetermined angle of rotation). In such examples, the set point (i.e., the predetermined angle of rotation) pressure would be determined by sensing at least the position of the subject in real time.

If the control system 12 determines that the new control signal did not cause rotation of the subject into the predetermined angle of rotation set for the continuous lateral rotation therapy routine, for example, via comparison, the control system 12 updates the database to indicate that the new control signal did not result in the predetermined angle of rotation, and then the control system 12 utilizes the algorithm to calculate a yet another control signal to further inflate and/or deflate the right rotation bladder 48 and/or the left rotation bladder 50 an amount sufficient to move the subject into the predetermined angle of rotation as specified in the continuous lateral rotation therapy routine, during which the control system 12 monitors the subject via the one or more sensors 53. If the control system 12 determines that the yet another control signal resulted in rotation of the subject into the predetermined angle of rotation set for the continuous lateral rotation therapy routine, for example, via comparison, the control system 12 updates the database to indicate the same and continues with the continuous lateral rotation therapy routine. However, the feedback loop continues to iteratively correct the movement of the subject into the predetermined angle of rotation if the control system 12 determines that the yet another control signal did not cause rotation of the subject into the predetermined angle of rotation set for the continuous lateral rotation therapy routine.

The control system 12 may also track position of the subject on the person support apparatus 10 via the one or more sensors 53. When the subject is in either the left or right rotation position and as the subject is being moved into either such rotation position, the one or more sensors 53 may measure a distance between the subject and either of the side rails 28, 30 and transmit such data indicative of such distance to the electrical control system 42. If the control system 12 determines that the distance between the subject and either of the side rails 28, 30 is less than a minimum distance, which may be indicative that the subject is sliding or shifting in position, the control system 12 may generate an alert to the caregiver and/or cease movement of the person support apparatus 10. Similarly, when the subject is in the flat orientation, if the control system 12 determines that the subject is less than the minimum distance to either of the side rails 28, 30, the control system 12 may generate an alert to the caregiver and/or cease any subsequent movement of the person support apparatus 10 until the caregiver has properly repositioned the subject. Thus, if the distance between the subject and either of the side rails 28, 30 is changing in a way that indicates that the subject was rolling towards either of the side rails 28, 30, the control system 12 may have a safety feature or mode (e.g., similar to a CPR mode whereby the person support apparatus is automatically repositioned to facility the administration of CPR and/or other resuscitation efforts) that would deflate the right rotation bladder 48 and/or the left rotation bladder 50.

In embodiments, the control system 12 optimizes continuous lateral rotation therapy based on physiological data or biomarkers. For example, the one or more sensors 53 may monitor SpO2 of the subject and transmit SpO2 data indicative of the subject's SpO2 to the control system 12 as the subject is moved from the flat orientation to left rotation position or the right rotation position. If the control system 12 determines that the subject's measured SpO2 is outside a predetermined SpO2 range when in any particular angle of rotation, the control system 12 may log the particular angle at which the SpO2 measurement departed from the predetermined SpO2 range. Also, if the control system 12 determines that the subject's measured SpO2 is outside the predetermined range, the control system 12 may cease movement of the person support apparatus 10 and/or generate an alert to the caregiver. For example, as the control system 12 causes movement into the left rotation position (or the right rotation position), the control system 12 may cease any further movement into the left rotation position (or the right rotation position) upon determining that the subject's measured SpO2 is outside the predetermined SpO2 range. The control system 12 may log the angle of rotation at which the subject's measured SpO2 departed the predetermined range in the database, and the control system 12 may subsequently utilize this data during continuous lateral rotation therapy to ensure that the subject's SpO2 remains within the predetermined range. For example, if the subject is supposed to undergo a continuous lateral rotation therapy routine on the person support apparatus 10, during which the subject will be held at certain angles of rotation, the control system 12 may reference the database to determine whether the subject's SpO2 has fallen outside the predetermined range when held at the certain angles of rotation and, if so, the control system 12 may establish a new predetermined angle of rotation where the subject's SpO2 is within the predetermined SpO2 range. In embodiments, if the continuous lateral rotation therapy routine establishes certain durations of time that the subject is held at certain angles of rotations, the control system 12 may reference the database to determine whether the subject's SpO2 has fallen or risen outside the predetermined range of SpO2 when held at the certain angles of rotation and to determine how long it took for the subject's SpO2 to depart from the predetermined SpO2 range. Here, the control system 12 may establish a new duration of time (e.g., a shorter duration of time) at which the subject is held at the certain angle of rotation to ensure that the subject's SpO2 is maintained within the predetermined SpO2 range when the subject is held at the certain angle of rotation, and then the control system 12 may move the subject into a new angle of rotation or the flat orientation before their SpO2 departs the predetermined SpO2 range. In embodiments, the control system 12 could utilize SpO2 data as part of a safety feature, for example, if the subject's measured SpO2 drops below a certain threshold value (e.g., set by a clinician), the continuous lateral rotation therapy could be immediately stopped and/or the an alert may be generated. In embodiments, physiological data or biomarkers other than SpO2 may be utilized, in combination with SpO2 or in lieu thereof, as described herein to optimize continuous lateral rotation therapy, such as skin degradation, pulmonary status, etc. Not only could the foregoing physiological data and/or biomarkers be utilized to optimize continuous lateral rotation therapy as described above, but such data may provide numerous clinical benefits. For example, such data could be useful for researching how to best serve subjects with surface-based pulmonary therapies.

In embodiments, the control system 12 calculates a weight distribution of the subject based on height and/or weight data captured by the sensors 53. Here, the database may include weight distribution data from other, previous subjects and the control signals utilized to position those other subjects into the predetermined angles of rotation based on their weight distribution. Using this database, the control system 12 compare the subject's weight distribution data with that of the previous subjects, and then the control system 12 may choose a baseline algorithm based weight distribution data from previous subjects that have similar weight distribution as the current subject. Then, as the subject undergoes a continuous lateral rotation therapy routine driven by the baseline algorithm, data about whether the subject is actually moved into the predetermined angles of rotation based on the control signals may be stored in the database and the algorithm may be updated with such newly captured subject specific data.

The control system 12 employs a computing model to build an ever expanding database to more accurately build the algorithm and calculate the control signals for inflating and/or deflating the right rotation bladder 48 and/or the left rotation bladder 50. The computing model is iterative and may continue until the subject is properly positioned in the predetermined angle of rotation as specified in the continuous lateral rotation therapy routine, such the control system 12 may learn. As described herein, the computing model can be a machine learning model and can be trained to identify control signals for the bladder system 47 based on training data, such as the database detailed herein. As used herein, the term “machine learning model,” and its equivalents, can refer to a computer-based model configured to identify patterns in data, and which can improve its pattern recognition based on training data. For example, the computing model can include at least one of a deep learning model, a linear regression model, a logistic regression model, a gradient boost machine, or some other machine learning model. The training data may comprise a database of continuous lateral rotation therapy routines, including the subject specific database and/or the shared characteristic database described herein. In various cases, the training data can include sensor data from the one or more sensors 53. The training data may also include EMR data from multiple EMRs, which may be associated with multiple subjects (including or excluding the subject). The training data may also indicate whether those subjects responded satisfactorily to the continuous lateral rotation therapy routine. Using the training data, one or more numerical factors of the computing model can be optimized to accurately predict an amount of inflation or deflation the right rotation bladder 48 and/or the left rotation bladder 50. This process of optimization can be referred to as “training.” Once trained, the computing model can be configured to accurately output control signals for inflating and/or deflating the right rotation bladder 48 and/or the left rotation bladder 50, based on inputs including the particular subject undergoing the therapy routine, whether the subject has certain shared characteristics, and/or a position of the subject, which may be indicated by data from the one or more sensors 53 and/or the EMR.

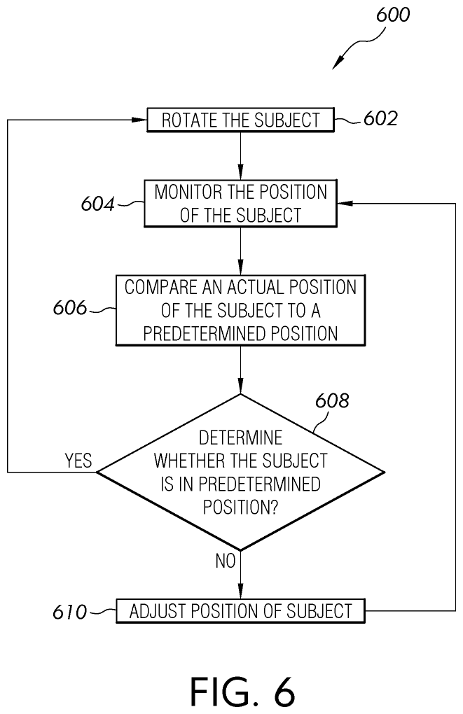

FIG. 6 illustrates an example process 600 for controlling continuous lateral rotation therapy, according to one or more embodiments of the present disclosure. The process 600 can be performed by an apparatus or entity, such as the person support apparatus 10 with the control system 12 described herein. For example, the process 600 can be performed by one or more computing devices including memory that stores instructions and one or more processors that, when executing the instructions, perform the operations of the process 600.

At block 602, the entity moves or rotates the subject pursuant to a continuous lateral rotation therapy routine. The continuous lateral rotation therapy routine comprises at least one predetermined turn angle and a period of time at which the subject is to be maintained at the predetermined turn angle, and the continuous lateral rotation therapy routine is stored in the memory device 64. As previously mentioned, the predetermined turn angle is a predetermined position that a subject is expected to be moved into when undergoing a continuous lateral rotation therapy routine. The routine may be a pre-programmed routine that is stored in the memory device 64 or the routine may be comprised of user input entered via the user interface 40 and which may thereafter be stored in the memory device 64. Also, control signals associated with inflating/deflating the right rotation bladder 48 and/or the left rotation bladder 50 into the predetermined turn angle of the routine are compiled in the database that is stored in the memory device 64. Here, the operator or caregiver initiates a continuous lateral rotation therapy routine, thereby causing the control system 12 to transmit a control signal associated with the predetermined turn angle of the initiated continuous lateral rotation therapy routine, which in turn causes the person support apparatus 10 to rotate or move the subject.

At block 604, the entity monitors the position of the subject as they are moved into the predetermined turn angle. Here, the one or more sensors 53 may capture data about the subject's actual position as they are moved into the predetermined turn angle. In embodiments, the one or more sensors 53 monitor the subject while the subject is moving, whereas, in other embodiments, the one or more sensors 53 capture data indicative of the subject's position after the subject has been moved according to the transmitted control signal. The entity may utilize that sensed data to determine an actual position into which the subject was moved as a result of the control signal.

At block 606, the entity compares the actual position of the subject to a predetermined position. In particular, the entity compares the actual position into which the subject was moved (at block 602) to the predetermined position, wherein the predetermined position may be part of the pre-programmed routine that is stored in the memory device 64 or user input entered via the user interface 40 and stored in the memory device 64.

Then, at block 608, the entity determines whether the subject is in the predetermined position. Stated differently, the entity determines whether the control signal resulted in proper positioning of the subject. If the entity determines, at block 608, that the actual position of the subject is equal to the predetermined position (i.e., that the subject is in the predetermined position), the entity may continue running the continuous lateral rotation therapy routine. For example, if the entity determines that the subject is in the predetermined position, the entity may continue running the continuous lateral rotation therapy routine by maintaining the subject in that position for the period of time set for the routine, and then moving the subject into a new position set for the routine, as indicated at block 602, etc.

At block 610, the entity adjusts the position of the subject if it is determined, at block 608, that the actual position of the subject is not equal to the predetermined position. The entity may adjust position of the subject by transmitting a correction control signal that will move the subject into the predetermined position. The correction control signal may be associated with the difference between the actual position of the subject and the predetermined position. However, as the entity adjusts the position of the subject based on the correction control signal, the entity monitors the position of the subject, as indicated at block 604, and compares (at block 606) the actual position into which the subject was moved (at block 602) via the correction control signal to the predetermined position.

If the entity determines, at block 608, that the actual position of the subject is equal to the predetermined position (i.e., that the correction control signal moved the subject into the predetermined position), the entity may update the database to reflect the fact that the subject was actually moved into the predetermined position by both the (original) control signal and the correction control signal. Also, the entity may calculate a new control signal based on both the (original) control signal and the correction control signal that will result in actual positioning of the subject in the predetermined position. For example, if the subject weighed W pounds and the algorithm predicted that an amount of pressure P was needed to rotate (that particular subject) X degrees (i.e., into the predetermined angle of rotation) but the subject was not actually moved X degrees (into the predetermined angle of rotation), the algorithm would be changed or updated to reflect that the amount of pressure P plus or minus pressure P′ (P+/−P′) was needed to move the subject X degrees, such that the algorithm would next time direct a new amount of pressure to move that particular subject (or subjects of equal/similar weight) X degrees (into the predetermined angle of rotation) where such new amount of pressure is equal to P plus or minus pressure P′ (P+/−P′). Thus, when that same subject (or a subject having shared characteristics with that subject) later on undergoes continuous lateral rotation therapy routine where they are to be moved into the predetermined position, the entity may transmit the new control signal to actually position the subject into the predetermined position. The entity may then continue running the continuous lateral rotation therapy routine (i.e., by maintaining the subject in that position for the period of time set for the routine, and then moving the subject into a new position set for the routine, as shown at block 602, etc.).

If it is determined, at block 608, that the correction control signal did not move the subject into the predetermined position, then the entity may readjust the position of the subject, as indicated at block 610. The entity may readjust position of the subject by transmitting a second correction control signal that will move the subject into the predetermined position. The second correction control signal may be associated with the difference between the actual position of the subject resulting from the correction control signal and the predetermined position. However, as mentioned above, the entity monitors the position of the subject, as indicated at block 604, as the entity readjusts the position of the subject based on the second correction control signal, and the entity compares, as indicated at block 606, the actual position into which the subject was adjusted (at block 610) via the second correction control signal to the predetermined position. Thus, the process 600 is iterative and the entity may continue performing its steps until the subject is properly positioned in the predetermined position, and the database may be updated with the new control signals that will result in such proper positioning.

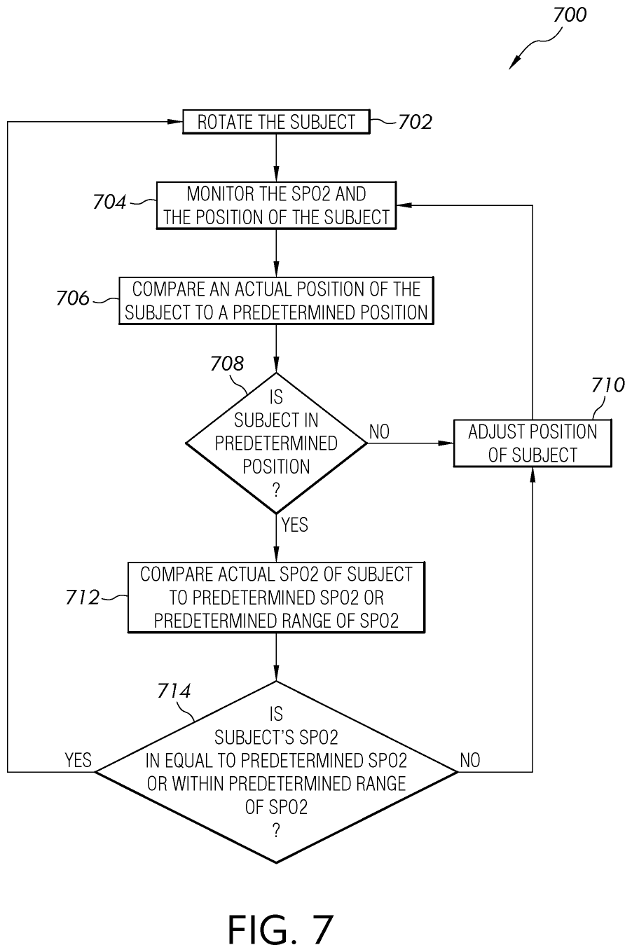

FIG. 7 illustrates another example process 700 for controlling continuous lateral rotation therapy, according to one or more embodiments of the present disclosure. While the process 600 of FIG. 6 automatically determined a positioning of the subject on the person support apparatus 10 based on that positioning, the process 700 controls continuous lateral rotation therapy based on oxygen saturation of a subject on a person support apparatus 10. Thus, in these examples, at least one of the sensors 53 is configured to measure SpO2 of the subject and at least one of the sensors 53 is configured to detect an actual angle of rotation of the subject. The process 700 can be performed by an apparatus or entity, such as the person support apparatus 10 with the control system 12 described herein. For example, the process 700 can be performed by one or more computing devices including memory that stores instructions and one or more processors that, when executing the instructions, perform the operations of the process 700.

At block 702, the entity moves or rotates the subject pursuant to a continuous lateral rotation therapy routine. Here, the continuous lateral rotation therapy routine comprises at least one predetermined turn angle, a period of time at which the subject is to be maintained at the predetermined turn angle, and at least one predetermined range of SpO2 values that the subject should exhibit when maintained at the predetermined turn angle for the period of time; and this continuous lateral rotation therapy routine is stored in the memory device 64. As previously mentioned, the predetermined turn angle is a predetermined position that a subject is expected to be moved into when undergoing a continuous lateral rotation therapy routine. The routine may be a pre-programmed routine that is stored in the memory device 64 or the routine may be comprised of user input entered via the user interface 40 and which may thereafter be stored in the memory device 64. Also, control signals associated with inflating/deflating the right rotation bladder 48 and/or the left rotation bladder 50 into the predetermined turn angle of the routine are compiled in the database that is stored in the memory device 64. Here, the operator or caregiver initiates a continuous lateral rotation therapy routine, thereby causing the control system 12 to transmit a control signal associated with the predetermined turn angle of the initiated continuous lateral rotation therapy routine, which in turn causes the person support apparatus 10 to rotate or move the subject.

At block 704, the entity monitors the SpO2 and the position of the subject as they are moved into the predetermined turn angle. Here, a first of the one or more sensors 53 monitors the subject's SpO2 and generates SpO2 data indicative of the same, whereas a second of the one or more sensors 53 monitors the position of the subject and generates data indicative of the same. In embodiments, the one or more sensors 53 monitor the subject while the subject is moving, whereas, in other embodiments, the one or more sensors 53 capture data indicative of the subject's position after the subject has been moved according to the transmitted control signal.

At block 706, the entity compares the actual position of the subject to a predetermined position. In particular, the entity compares the actual position into which the subject was moved (at block 702) to the predetermined position. The predetermined position may be part of the pre-programmed routine that is stored in the memory device 64 or user input entered via the user interface 40 and stored in the memory device 64.

Then, at block 708, the entity determines whether the subject is in the predetermined position. Stated differently, the entity determines at block 708 whether the control signal resulted in proper positioning of the subject. If the entity determines, at block 708, that the actual position of the subject is not equal to the predetermined position (i.e., that the subject is not in the predetermined position), the entity adjusts the position of the subject as indicated at block 710. The entity may adjust position of the subject by transmitting a correction control signal that will move the subject into the predetermined position. The correction control signal may be associated with the difference between the actual position of the subject and the predetermined position. However, as the entity adjusts the position of the subject based on the correction control signal (at the block 710), the entity monitors the position and the SpO2 of the subject, as indicated at block 704, and compares (at block 706) the actual position into which the subject was moved (at block 710) via the correction control signal to the predetermined position.

If the entity determines, at block 708, that the actual position of the subject is equal to the predetermined position (i.e., that the subject is in the predetermined position), the entity may continue running the continuous lateral rotation therapy routine (i.e., by maintaining the subject in that position for the period of time set for the routine, and then moving the subject into a new position set for the routine, as shown at block 702, etc.).

The entity also compares, at block 712, the actual SpO2 of the subject monitored by first one or more sensors 53 with the predetermined SpO2 or predetermined range of SpO2 values. This comparison (at block 712) may occur simultaneously as the entity compares the actual position into which the subject was moved with the predetermined position (at block 706), or it may occur after the entity determines (at block 708) that the subject has been moved into predetermined position. If the entity determines, at block 714, that the subject's actual SpO2 is equal to the predetermined SpO2 or within the predetermined SpO2 range (i.e., that the subject's SpO2 is acceptable), the entity may continue running the continuous lateral rotation therapy routine (i.e., by maintaining the subject in that position for the period of time set for the routine, and then moving the subject into a new position set for the routine, as shown at block 702, etc.).

Also, if the entity determines, at block 714, that the subject's actual SpO2 is equal to the predetermined SpO2 or within the predetermined SpO2 range, the entity may update the database to reflect the fact that, when moved into the predetermined angle of rotation, the subject's SpO2 was within the predetermined SpO2 range or equal to the predetermined SpO2. Moreover, the entity may update the database to reflect the fact that the subject's SpO2 was within the predetermined SpO2 range or equal to the predetermined SpO2 for the for the period of time at which the subject was maintained in the predetermined position. Further, the entity may update the database to reflect the fact that the control signal or the corrected control signal maintained the subject's SpO2 within the predetermined SpO2 range or at a value equal to the predetermined SpO2. The entity may then continue running the continuous lateral rotation therapy routine (i.e., by maintaining the subject in that position for the period of time set for the routine, and then moving the subject into a new position set for the routine, as shown at block 702, etc.).

If it is determined, at block 714, that the subject's actual SpO2 is not equal to the predetermined SpO2 or not within the predetermined SpO2 range, then the entity may readjust the position of the subject, as indicated at 710, and thereby move the subject into a new predetermined angle of rotation. The entity may update the database to reflect the fact that the subject's SpO2 was not within the predetermined SpO2 range or not equal to the predetermined SpO2 when moved into the predetermined position. Also, if the subject's SpO2 is initially equal to the predetermined SpO2 or within the predetermined SpO2 range when the subject is moved into the predetermined position, but then later changes (i.e., to be not equal to the predetermined SpO2 or not within the predetermined SpO2 range) during the period of time at which they are held in the predetermined positon, the entity may readjust the position of the subject, as indicated at block 710, into a new predetermined angle of rotation. The entity may update the database to reflect the fact that the subject's SpO2 was not within the predetermined SpO2 range or not equal to the predetermined SpO2 for the for the period of time at which the subject was maintained in the predetermined position. Moreover, the entity may update the database with the amount of time at which the subject was maintained in the predetermined position before their SpO2 drifted off of the acceptable value or out of the acceptable range.

The entity may update the database to reflect the fact that the control signal or the corrected control signal caused the subject's SpO2 to drift outside of the predetermined SpO2 range or drift off of the predetermined SpO2 value. The entity may then calculate a new control signal based on both the (original) control signal and the correction control signal that will move the subject into the new predetermined angle of rotation where the subject's SpO2 is maintained within the predetermined SpO2 range or equal to the predetermined SpO2. The entity may then use that new control signal to maintain the subject's SpO2 as desired when moving the subject or adjusting position of the subject as described herein.

For example, when adjusting position of the subject, as indicated at block 710, the entity may utilize the new control signal to thereby move the subject into the new predetermined angle of rotation. As the subject is moved, the subject's actual position is monitored via the one or more sensors 53, as indicated at block 704, and the subject actual position is compared to the new predetermined angle of rotation to determine whether they have been moved into the new predetermined angle of rotation, as indicated at block 706. Also as the subject is moved into the new predetermined angle of rotation via the new control signal, the subject's SpO2 is monitored via the one or more sensors 53, as indicated at block 704, to determine whether their SpO2 is within the predetermined SpO2 range or equal to the predetermined SpO2, as indicated at block 714. The entity may update the database as to whether the subject's SpO2 drifted off of the acceptable SpO2 value or out of the acceptable range of SpO2 when the subject was moved using the new control signal, or, after being moved into the new predetermined angle of rotation, whether the subject's SpO2 thereafter drifted off of the acceptable SpO2 value or out of the acceptable range of SpO2 as the subject was maintained in the new predetermined position for the period of time.

Accordingly, the entity is operable to adjust the predetermined angle of rotation at which the subject is held based on the SpO2 data received from the one or more sensors 53. In embodiments, the entity compares the actual SpO2 of the subject with the predetermined SpO2 value or predetermined range of SpO2 values only after the subject has been properly positioned in the predetermined angle of rotation. In other embodiments, the entity constantly compares the actual SpO2 of the subject with the predetermined SpO2 or predetermined range of SpO2 values at all times during the continuous lateral rotation therapy, for example, as the subject is being moved. In embodiments, the entity ceases further movement of the subject if it determines that subject's SpO2 is outside the predetermined SpO2 range or not equal to the predetermined SpO2. In embodiments, the entity is operable to adjust a duration of time at which the subject is held in the predetermined angle of rotation based on the SpO2 of the subject as monitored by the one or more sensors 53.

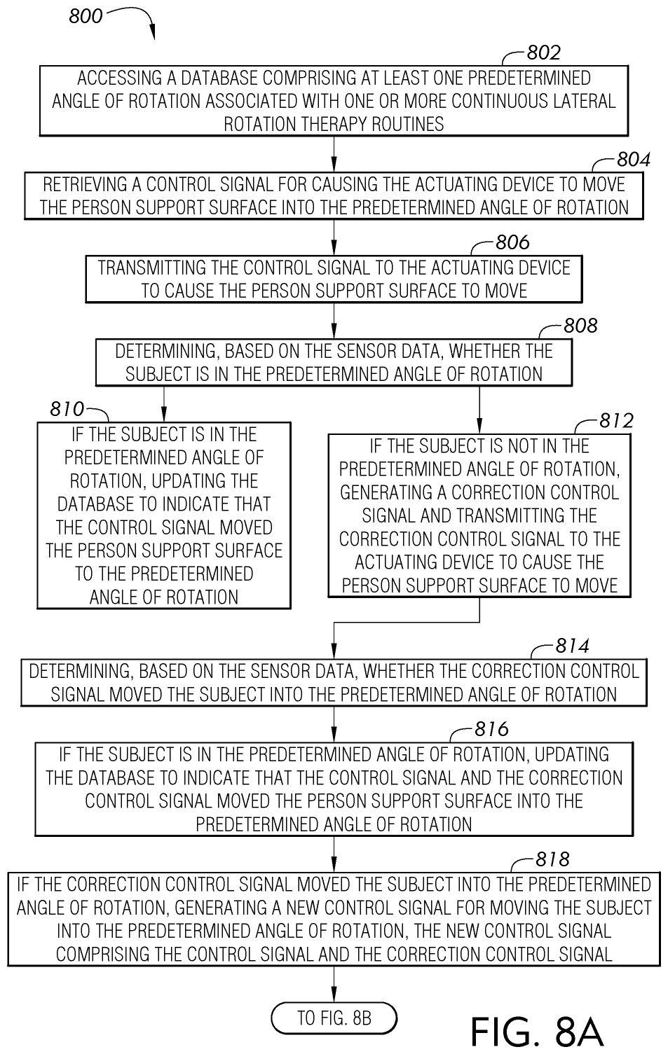

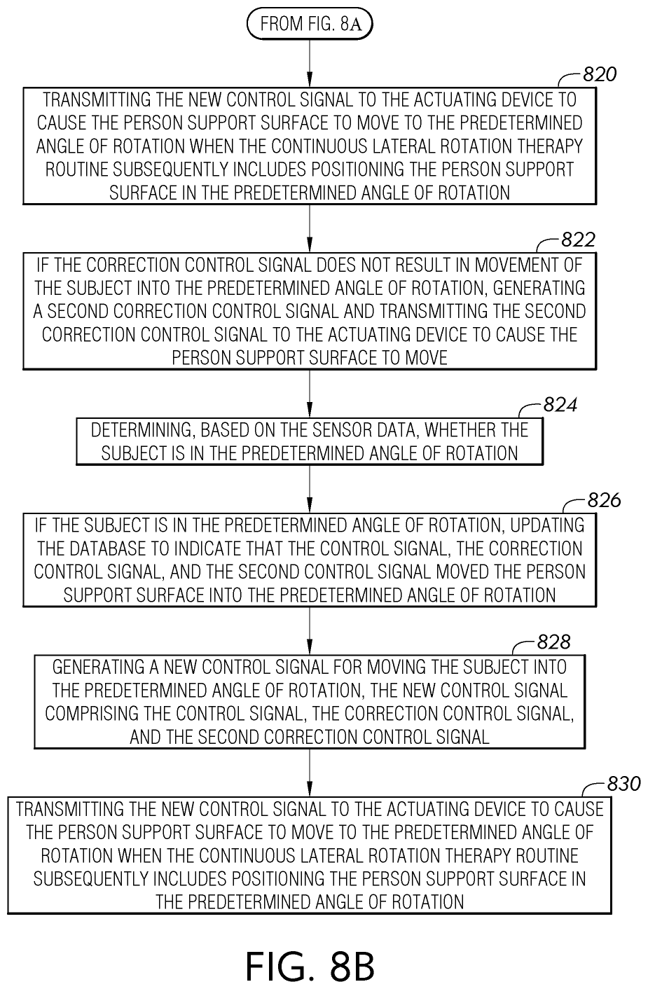

FIGS. 8A-8B illustrate an example method 800 for controlling a person support, apparatus, according to one or more embodiments of the present disclosure. In the illustrated embodiment, the method 800 includes, at block 802, accessing a database comprising at least one predetermined angle of rotation associated with one or more continuous lateral rotation therapy routines and, at block 804, retrieving a control signal for causing the actuating device to move the person support surface into the predetermined angle of rotation. The method 800 also includes, at block 806, transmitting the control signal to the actuating device to cause the person support surface to move.

Then, at block 808, the method 800 includes determining, based on the sensor data, whether the subject is in the predetermined angle of rotation. If the subject is in the predetermined angle of rotation, the method 800 includes updating the database to indicate that the control signal moved the person support surface to the predetermined angle of rotation, as indicated at block 810.

However, if the subject is not in the predetermined angle of rotation, the method 800 may include generating a correction control signal and transmitting the correction control signal to the actuating device to cause the person support surface to move, as indicated at block 812. The method 800 may then include determining, based on the sensor data, whether the correction control signal moved the subject into the predetermined angle of rotation, as indicated at block 814. Then, if the subject is in the predetermined angle of rotation, the method 800 may include updating the database to indicate that the control signal and the correction control signal moved the person support surface into the predetermined angle of rotation, as indicated at block 816.

At block 818, if the correction control signal moved the subject into the predetermined angle of rotation, the method 800 may include generating a new control signal for moving the subject into the predetermined angle of rotation, the new control signal comprising the control signal and the correction control signal. Then, at block 820, the method 800 may include transmitting the new control signal to the actuating device to cause the person support surface to move to the predetermined angle of rotation when the continuous lateral rotation therapy routine subsequently includes positioning the person support surface in the predetermined angle of rotation.

If, at block 814, the correction control signal does not result in movement of the subject into the predetermined angle of rotation, the method 800 may include generating a second correction control signal and transmitting the second correction control signal to the actuating device to cause the person support surface to move, as indicated at block 822. Then, at block 824, the method may include determining, based on the sensor data, whether the subject is in the predetermined angle of rotation.

At block 826, if the subject is in the predetermined angle of rotation, the method 800 may include updating the database to indicate that the control signal, the correction control signal, and the second control signal moved the person support surface into the predetermined angle of rotation. Then, the method 800 may include generating a new control signal for moving the subject into the predetermined angle of rotation, the new control signal comprising the control signal, the correction control signal, and the second correction control signal, as indicated at block 828, and transmitting the new control signal to the actuating device to cause the person support surface to move to the predetermined angle of rotation when the continuous lateral rotation therapy routine subsequently includes positioning the person support surface in the predetermined angle of rotation, as indicated at block 830.

In embodiments where the one or more sensors 53 are configured to measure SpO2 of the subject and generate SpO2 data, the method 800 may further include adjusting the predetermined angle of rotation at which the subject is held based on the SpO2 data, and/or adjusting a duration of time at which the subject is held in the predetermined angle of rotation based on the SpO2 data.

Further aspects of the embodiments described herein are provided by the subject matter of the following clauses:

Clause 1. A person support apparatus, comprising: a person support surface configured to support a subject, the person support surface including an actuating device for rotating the person support surface from a flat orientation to a rotated position; one or more sensors configured to detect one or more parameters of the subject and the person support surface and generate sensor data based on the one or more parameters thereof; and a controller operable to: access a database comprising at least one predetermined angle of rotation associated with one or more continuous lateral rotation therapy routines; retrieve a control signal for causing the actuating device to move the person support surface into the predetermined angle of rotation; transmit the control signal to the actuating device to cause the person support surface to move; determine, based on the sensor data, whether the subject is in the predetermined angle of rotation; if the subject is in the predetermined angle of rotation, update the database to indicate that the control signal moved the person support surface to the predetermined angle of rotation; if the subject is not in the predetermined angle of rotation, generate and transmit a correction control signal to the actuating device to cause the person support surface to move; determine, based on the sensor data, whether the correction control signal moved the subject into the predetermined angle of rotation; and if the subject is in the predetermined angle of rotation, update the database to indicate that the control signal and the correction control signal moved the person support surface into the predetermined angle of rotation.

Clause 2. The person support apparatus of clause 1, wherein the controller is further operable to: if the correction control signal moved the subject into the predetermined angle of rotation, generate a new control signal for moving the subject into the predetermined angle of rotation, the new control signal comprising the control signal and the correction control signal; and transmit the new control signal to the actuating device to cause the person support surface to move to the predetermined angle of rotation when the continuous lateral rotation therapy routine subsequently includes positioning the person support surface in the predetermined angle of rotation.

Clause 3. The person support apparatus of any of the preceding clauses, wherein, when the correction control signal does not result in movement of the subject into the predetermined angle of rotation, the instructions, when executed by the processor, further cause the processor to: generate and transmit a second correction control signal to the actuating device to cause the person support surface to move; determine, based on the sensor data, whether the subject is in the predetermined angle of rotation; and if the subject is in the predetermined angle of rotation, update the database to indicate that the control signal, the correction control signal, and the second control signal moved the person support surface into the predetermined angle of rotation; generate a new control signal for moving the subject into the predetermined angle of rotation, the new control signal comprising the control signal, the correction control signal, and the second correction control signal; and transmit the new control signal to the actuating device to cause the person support surface to move to the predetermined angle of rotation when the continuous lateral rotation therapy routine subsequently includes positioning the person support surface in the predetermined angle of rotation.

Clause 4. The person support apparatus of any of the preceding clauses, wherein the rotation position comprises either a left rotation position or a right rotation position.

Clause 5. The person support apparatus of any of the preceding clauses, wherein the one or more sensors comprises a load beam or load cell configured to detect a weight the subject, an infrared camera configured to generate at least one infrared image of the subject, a camera configured to generate at least one image of the subject, and/or a video camera configured to generate at least one video of the subject, and combinations thereof.

Clause 6. The person support apparatus of any of the preceding clauses, wherein the person support surface includes a head end, a foot end, and a pair of side rails extending between the head end and the foot end, wherein the one or more sensors are attached to either or both of side rails.

Clause 7. The person support apparatus of any of the preceding clauses, wherein the person support surface includes a head end, a foot end, and a pair of side rails extending between the head end and the foot end, wherein the one or more sensors are attached to either or both of the head end and the foot end.

Clause 8. The person support apparatus of any of the preceding clauses, wherein the actuating device comprises a pneumatic control system coupled to at least one bladder.

Clause 9. The person support apparatus of clause 8, wherein the at least one bladder comprises a left rotation bladder and a right rotation bladder.

Clause 10. The person support apparatus of any of the preceding clauses, further comprising a user interface that comprises one or more left rotation buttons and one or more right rotation buttons for entering parameters of the rotation position.

Clause 11. The person support apparatus of any of the preceding clauses, wherein the controller is configured to determine an identity of the subject via the sensor data.

Clause 12. The person support apparatus of clause 11, wherein a determination of whether the person support surface has been moved into the predetermined angle of rotation is based on data associated with the identity of the subject.

Clause 13. The person support apparatus of any of the preceding clauses, wherein the controller is configured to determine one or more physical or physiological characteristics of the subject via the sensor data.

Clause 14. The person support apparatus of clause 13, wherein a determination of whether the person support surface has been moved into the predetermined angle of rotation is based on data associated with the one or more physical or physiological characteristics of the subject.

Clause 15. The person support apparatus of any of the preceding clauses, wherein the controller is configured to communicate with an EMR system.

Clause 16. The person support apparatus of clause 15, wherein the controller is configured to determine an identity of the subject via EMR data retrieved from the EMR system.

Clause 17. The person support apparatus of clause 15, wherein the controller is configured to update the EMR system with updated data captured by the one or more sensors.

Clause 18. The person support apparatus of any of the preceding clauses, wherein the controller is configured to determine a distance between the subject and a side of the person support surface via the sensor data.

Clause 19. The person support apparatus of, clause 18 wherein, if the controller determines that the distance between the subject and the side is less than a minimum distance, the controller generates an alert and/or ceases movement of the person support surface.

Clause 20. The person support apparatus of any of the preceding clauses, wherein the memory device stores at least one continuous lateral therapy routine comprising a plurality of angles of rotation, wherein the plurality of angles of rotation includes the predetermined angle of rotation.

Clause 21. The person support apparatus of any of the preceding clauses, wherein the person support surface includes a subject contact surface comprising a hydro-chromic polymer layer that changes colors when contacted by an extraneous material.

Clause 22. The person support apparatus of clause 21, wherein the hydro-chromic polymer layer is provided on the subject contact surface as an ink or as a film.

Clause 23. The person support apparatus of any of the preceding clauses, wherein the person support surface includes a base subject contact surface layer having a base color and a top subject contact surface layer provided over the base subject contact surface layer to cover the base color, wherein, when the subject abrades the top subject contact surface layer, the base color of the base subject contact surface layer is exposed.

Clause 24. The person support apparatus of clause 23, wherein the top subject contact surface layer comprises a polymer coating.

Clause 25. The person support apparatus of any of the preceding clauses, wherein the one or more sensors are configured to measure SpO2 of the subject and generate SpO2 data, and the controller is operable to adjust the predetermined angle of rotation at which the subject is held based on the SpO2 data.

Clause 26. The person support apparatus of any of the preceding clauses, wherein the controller is operable to adjust a duration of time at which the subject is held in the predetermined angle of rotation based on the SpO2 data.

Clause 27. A person support apparatus comprising: a person support surface configured to support a subject, the person support surface including an actuating device for rotating the person support surface, from a flat orientation to either a rotation position; at least one first sensor configured to detect SpO2 of the subject and to generate SpO2 data; at least one second sensor configured to detect an actual angle of rotation of the subject and generate data indicative of the actual angle of rotation of the subject; and a controller operable to control the actuating device, the controller comprising a processor and a non-transitory memory device, the memory device storing: a database comprising at least one predetermined angle of rotation associated with one or more continuous lateral rotation therapy routines and comprising at least one predetermined SpO2 range; and instructions that, when executed by the processor, cause the processor to: determine, based on the database, a control signal that will cause the actuating device to move the person support surface into the predetermined angle of rotation; transmit the control signal to the actuating device to thereby move the person support surface; determine, based on the data indicative of the actual angle, whether the subject is in the predetermined angle of rotation; determine, based on the SpO2 data, whether the subject is within the predetermined SpO2 range; and update the database to indicate whether, when moved into the predetermined angle of rotation, the subject was within the predetermined SpO2 range.

Clause 28. The person support apparatus of clause 27, wherein, if the control signal moved the subject into the predetermined angle of rotation but, when in the predetermined angle of rotation, the subject is not within the predetermined SpO2 range, the instructions, when executed by the processor, further cause the processor to: determine, based on the database, a new control signal that will cause the actuating device to move the person support surface into a new predetermined angle of rotation; transmit the new control signal to the actuating device to thereby move the person support surface; determine, based on the data indicative of an actual angle, whether the subject is in the new predetermined angle of rotation; determine, based on the SpO2 data, whether the subject is within the predetermined SpO2 range when in the new predetermined angle of rotation; and update the database to indicate whether, when moved into the new predetermined angle of rotation via the new control signal, the subject was within the predetermined SpO2 range.

Clause 29. The person support apparatus of any of the preceding clauses, wherein the controller is operable to adjust the predetermined angle of rotation at which the subject is held based on the SpO2 data.

Clause 30. The person support apparatus of any of the preceding clauses, wherein the controller is operable to adjust a duration of time at which the subject is held in the predetermined angle of rotation based on the SpO2 data.

Clause 31. The person support apparatus of any of the preceding clauses wherein the person support surface includes a subject contact surface comprising a hydro-chromic polymer layer that change colors when contacted by an extraneous material, and wherein the hydro-chromic polymer layer is provided on the subject contact surface as an ink or as a film.

Clause 32. The person support apparatus of any of the preceding clauses, wherein the person support surface includes a base subject contact surface layer having a base color and a top subject contact surface layer provided over the base subject contact surface layer to cover the base color, wherein, when the subject abrades the top subject contact surface layer, the base color of the base subject contact surface layer is exposed, and wherein the top subject contact surface layer comprises a polymer coating.

Clause 33. A controller for a person support apparatus, the person support apparatus comprising an actuating device for rotating the person support surface and one or more sensors configured to detect one or more parameters of a subject on the person support surface and generate sensor data based on the one or more parameters, the controller being operable to: access a database comprising at least one predetermined angle of rotation associated with one or more continuous lateral rotation therapy routines; retrieve a control signal for causing the actuating device to move the person support surface into the predetermined angle of rotation; transmit the control signal to the actuating device to cause the person support surface to move; determine, based on the sensor data, whether the subject is in the predetermined angle of rotation; if the subject is in the predetermined angle of rotation, update the database to indicate that the control signal moved the person support surface to the predetermined angle of rotation; if the subject is not in the predetermined angle of rotation, generate and transmit a correction control signal to the actuating device to cause the person support surface to move; determine, based on the sensor data, whether the correction control signal moved the subject into the predetermined angle of rotation; and if the subject is in the predetermined angle of rotation, update the database to indicate that the control signal and the correction control signal moved the person support surface into the predetermined angle of rotation.

Clause 34. The controller of clause 33, wherein the controller is further operable to: if the correction control signal moved the subject into the predetermined angle of rotation, generate a new control signal for moving the subject into the predetermined angle of rotation, the new control signal comprising the control signal and the correction control signal; and transmit the new control signal to the actuating device to cause the person support surface to move to the predetermined angle of rotation when the continuous lateral rotation therapy routine subsequently includes positioning the person support surface in the predetermined angle of rotation.

Clause 35. The controller of any of the preceding clauses, wherein, when the correction control signal does not result in movement of the subject into the predetermined angle of rotation, the instructions, when executed by the processor, further cause the processor to: generate and transmit a second correction control signal to the actuating device to cause the person support surface to move; determine, based on the sensor data, whether the subject is in the predetermined angle of rotation; and if the subject is in the predetermined angle of rotation, update the database to indicate that the control signal, the correction control signal, and the second control signal moved the person support surface into the predetermined angle of rotation; generate a new control signal for moving the subject into the predetermined angle of rotation, the new control signal comprising the control signal, the correction control signal, and the second correction control signal; and transmit the new control signal to the actuating device to cause the person support surface to move to the predetermined angle of rotation when the continuous lateral rotation therapy routine subsequently includes positioning the person support surface in the predetermined angle of rotation.

Clause 36. The controller of the preceding clauses, wherein the one or more sensors are configured to measure SpO2 of the subject and generate SpO2 data, and the controller is operable to: adjust the predetermined angle of rotation at which the subject is held based on the SpO2 data; and/or adjust a duration of time at which the subject is held in the predetermined angle of rotation based on the SpO2 data.

Clause 37. A method of controlling a person support apparatus, the person support apparatus comprising an actuating device for rotating the person support surface and one or more sensors configured to detect one or more parameters of a subject on the person support surface and generate sensor data based on the one or more parameters, the method comprising: accessing a database comprising at least one predetermined angle of rotation associated with one or more continuous lateral rotation therapy routines; retrieving a control signal for causing the actuating device to move the person support surface into the predetermined angle of rotation; transmitting the control signal to the actuating device to cause the person support surface to move; determining, based on the sensor data, whether the subject is in the predetermined angle of rotation; if the subject is in the predetermined angle of rotation, updating the database to indicate that the control signal moved the person support surface to the predetermined angle of rotation; if the subject is not in the predetermined angle of rotation, generating a correction control signal and transmitting the correction control signal to the actuating device to cause the person support surface to move; determining, based on the sensor data, whether the correction control signal moved the subject into the predetermined angle of rotation; and if the subject is in the predetermined angle of rotation, updating the database to indicate that the control signal and the correction control signal moved the person support surface into the predetermined angle of rotation.

Clause 38. The method of clause 37, further comprising: if the correction control signal moved the subject into the predetermined angle of rotation, generating a new control signal for moving the subject into the predetermined angle of rotation, the new control signal comprising the control signal and the correction control signal; and transmitting the new control signal to the actuating device to cause the person support surface to move to the predetermined angle of rotation when the continuous lateral rotation therapy routine subsequently includes positioning the person support surface in the predetermined angle of rotation.

Clause 39. The method of any of the preceding clauses, wherein, when the correction control signal does not result in movement of the subject into the predetermined angle of rotation, the instructions, when executed by the processor, the method further comprises: generating a second correction control signal and transmitting the second correction control signal to the actuating device to cause the person support surface to move; determining, based on the sensor data, whether the subject is in the predetermined angle of rotation; and if the subject is in the predetermined angle of rotation, updating the database to indicate that the control signal, the correction control signal, and the second control signal moved the person support surface into the predetermined angle of rotation; generating a new control signal for moving the subject into the predetermined angle of rotation, the new control signal comprising the control signal, the correction control signal, and the second correction control signal; and transmitting the new control signal to the actuating device to cause the person support surface to move to the predetermined angle of rotation when the continuous lateral rotation therapy routine subsequently includes positioning the person support surface in the predetermined angle of rotation.

Clause 40. The method of any of the preceding clauses, wherein the one or more sensors are configured to measure SpO2 of the subject and generate SpO2 data, and the further comprises: adjusting the predetermined angle of rotation at which the subject is held based on the SpO2 data; and/or adjusting a duration of time at which the subject is held in the predetermined angle of rotation based on the SpO2 data.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

What is claimed is:

1. A person support apparatus, comprising:
a person support surface configured to support a subject, the person support surface including an actuating device for rotating the person support surface from a flat orientation to a rotated position;
one or more sensors configured to detect one or more parameters of the subject and the person support surface and generate sensor data based on the one or more parameters thereof; and
a controller operable to:
access a database comprising at least one predetermined angle of rotation associated with one or more continuous lateral rotation therapy routines;
retrieve a control signal for causing the actuating device to move the person support surface into the predetermined angle of rotation;
transmit the control signal to the actuating device to cause the person support surface to move;
determine, based on the sensor data, whether the subject is in the predetermined angle of rotation;
if the subject is in the predetermined angle of rotation, update the database to indicate that the control signal moved the person support surface to the predetermined angle of rotation;
if the subject is not in the predetermined angle of rotation, generate and transmit a correction control signal to the actuating device to cause the person support surface to move;
determine, based on the sensor data, whether the correction control signal moved the subject into the predetermined angle of rotation; and
if the subject is in the predetermined angle of rotation, update the database to indicate that the control signal and the correction control signal moved the person support surface into the predetermined angle of rotation.

2. The person support apparatus of claim 1, wherein the controller is further operable to:
if the correction control signal moved the subject into the predetermined angle of rotation, generate a new control signal for moving the subject into the predetermined angle of rotation, the new control signal comprising the control signal and the correction control signal; and
transmit the new control signal to the actuating device to cause the person support surface to move to the predetermined angle of rotation when the continuous lateral rotation therapy routine subsequently includes positioning the person support surface in the predetermined angle of rotation.

3. The person support apparatus of claim 1, wherein, when the correction control signal does not result in movement of the subject into the predetermined angle of rotation, the instructions, when executed by the processor, further cause the processor to:
generate and transmit a second correction control signal to the actuating device to cause the person support surface to move;
determine, based on the sensor data, whether the subject is in the predetermined angle of rotation; and
if the subject is in the predetermined angle of rotation, update the database to indicate that the control signal, the correction control signal, and the second control signal moved the person support surface into the predetermined angle of rotation;
generate a new control signal for moving the subject into the predetermined angle of rotation, the new control signal comprising the control signal, the correction control signal, and the second correction control signal; and
transmit the new control signal to the actuating device to cause the person support surface to move to the predetermined angle of rotation when the continuous lateral rotation therapy routine subsequently includes positioning the person support surface in the predetermined angle of rotation.

4. The person support apparatus of claim 1, wherein the one or more sensors comprises a load beam or load cell configured to detect a weight the subject, an infrared camera configured to generate at least one infrared image of the subject, a camera configured to generate at least one image of the subject, and/or a video camera configured to generate at least one video of the subject, and combinations thereof.

5. The person support apparatus of claim 1, wherein the person support surface includes a head end, a foot end, and a pair of side rails extending between the head end and the foot end, wherein the one or more sensors are attached to at least one of the side rails, the head end, or the foot end.

6. The person support apparatus of claim 1, wherein the actuating device comprises a pneumatic control system coupled to at least one bladder.

7. The person support apparatus of claim 1, further comprising a user interface that comprises one or more left rotation buttons and one or more right rotation buttons for entering parameters of the rotation position.

8. The person support apparatus of claim 1, wherein:
the controller is configured to determine an identity of the subject via the sensor data, and
a determination of whether the person support surface has been moved into the predetermined angle of rotation is based on data associated with the identity of the subject.

9. The person support apparatus of claim 1, wherein:
the controller is configured to determine one or more physical or physiological characteristics of the subject via the sensor data, and
a determination of whether the person support surface has been moved into the predetermined angle of rotation is based on data associated with the one or more physical or physiological characteristics of the subject.

10. The person support apparatus of claim 1, wherein the controller is configured to:
determine an identity of the subject via EMR data retrieved from an EMR system; and
update the EMR system with updated data captured by the one or more sensors.

11. The person support apparatus of claim 1, wherein the controller is configured to determine a distance between the subject and a side of the person support surface via the sensor data, and if the controller determines that the distance between the subject and the side is less than a minimum distance, the controller generates an alert and/or ceases movement of the person support surface.

12. The person support apparatus of claim 1, wherein the memory device stores at least one continuous lateral therapy routine comprising a plurality of angles of rotation, wherein the plurality of angles of rotation includes the predetermined angle of rotation.

13. The person support apparatus of claim 1, wherein the person support surface includes a subject contact surface comprising a hydro-chromic polymer layer that changes colors when contacted by an extraneous material, the hydro-chromic polymer layer provided on the subject contact surface as an ink or as a film.

14. The person support apparatus of claim 1, wherein the person support surface includes a base subject contact surface layer having a base color and a top subject contact surface layer comprising a polymer coating and provided over the base subject contact surface layer to cover the base color, wherein, when the subject abrades the top subject contact surface layer, the base color of the base subject contact surface layer is exposed.

15. The person support apparatus of claim 1, wherein the one or more sensors are configured to measure SpO2 of the subject and generate SpO2 data, and the controller is operable to adjust the predetermined angle of rotation at which the subject is held based on the SpO2 data.

16. The person support apparatus of claim 1, wherein the controller is operable to adjust a duration of time at which the subject is held in the predetermined angle of rotation based on the SpO2 data.

17. A person support apparatus comprising:
a person support surface configured to support a subject, the person support surface including an actuating device for rotating the person support surface, from a flat orientation to either a rotation position;
at least one first sensor configured to detect SpO2 of the subject and to generate SpO2 data;
at least one second sensor configured to detect an actual angle of rotation of the subject and generate data indicative of the actual angle of rotation of the subject; and
a controller operable to control the actuating device, the controller comprising a processor and a non-transitory memory device, the memory device storing:
a database comprising at least one predetermined angle of rotation associated with one or more continuous lateral rotation therapy routines and comprising at least one predetermined SpO2 range; and
instructions that, when executed by the processor, cause the processor to:
determine, based on the database, a control signal that will cause the actuating device to move the person support surface into the predetermined angle of rotation;
transmit the control signal to the actuating device to thereby move the person support surface;
determine, based on the data indicative of the actual angle, whether the subject is in the predetermined angle of rotation;
determine, based on the SpO2 data, whether the subject is within the predetermined SpO2 range; and
update the database to indicate whether, when moved into the predetermined angle of rotation, the subject was within the predetermined SpO2 range.

18. The person support apparatus of claim 17, wherein, if the control signal moved the subject into the predetermined angle of rotation but, when in the predetermined angle of rotation, the subject is not within the predetermined SpO2 range, the instructions, when executed by the processor, further cause the processor to:
determine, based on the database, a new control signal that will cause the actuating device to move the person support surface into a new predetermined angle of rotation;
transmit the new control signal to the actuating device to thereby move the person support surface;
determine, based on the data indicative of an actual angle, whether the subject is in the new predetermined angle of rotation;
determine, based on the SpO2 data, whether the subject is within the predetermined SpO2 range when in the new predetermined angle of rotation; and
update the database to indicate whether, when moved into the new predetermined angle of rotation via the new control signal, the subject was within the predetermined SpO2 range.

19. The person support apparatus of claim 17, wherein the controller is operable to:
adjust the predetermined angle of rotation at which the subject is held based on the SpO2 data, and
adjust a duration of time at which the subject is held in the predetermined angle of rotation based on the SpO2 data.

20. The person support apparatus of claim 17, wherein the person support surface includes one of:
a subject contact surface comprising a hydro-chromic polymer layer that change colors when contacted by an extraneous material, and wherein the hydro-chromic polymer layer is provided on the subject contact surface as an ink or as a film, or
a base subject contact surface layer having a base color and a top subject contact surface layer provided over the base subject contact surface layer to cover the base color, wherein, when the subject abrades the top subject contact surface layer, the base color of the base subject contact surface layer is exposed, and wherein the top subject contact surface layer comprises a polymer coating.
